(19) United States
(12) Patent Application Publication
(10) Pub. No.: US 2024/0127073 A1
(43) Pub. Date: Apr. 18, 2024

(54) SYNTHETIC COGNITION (SC) INTUITIVE SYSTEM AND METHOD FOR PREDICTING, ANALYSING AND MANAGING COMPLEX ADAPTIVE AND NON-ADAPTIVE SYSTEMS IN ENGINEERED, CYBERNETIC OR PARTIALLY BIOLOGICAL APPLICATIONS

(71) Applicant: PAUL C SALINS, Bangalore (IN)

(72) Inventor: PAUL C SALINS, Bangalore (IN)

(21) Appl. No.: 18/257,722

(22) PCT Filed: Dec. 17, 2021
(86) PCT No.: PCT/IN2021/051185

§ 371 Date: Jun. 15, 2023

(30) Foreign Application Priority Data
Dec. 18, 2020 (IN) 202041055581

(51) Int. Cl. G06N 3/094; G06N 3/0464

(52) U.S. Cl. CPC G06N 3/094; G06N 3/0464

(57) ABSTRACT

The embodiments herein provide a system and method for managing complex adaptive and non-adaptive system using synthetic cognition/intuitive machine learning. In an embodiment the system captures broad outcomes in clearly identifiable categories in systems with a reverse approach to iteration without having to divide into small components for detailed analysis. The system divides a function into its parts to be able to juxtapose with other functions in various combinations to predict potential outcomes. The system generates a synthetic language representation of static/dynamic/continuum data, to be integrated into computational systems using a synthetic cognition language. The system captures a natural understanding of complex systems as a combination of distinct separate element.

### CROSS-REFERENCE TO RELATED APPLICATIONS

The present application is a National Phase Application of the Patent Cooperation Treaty (PCT) international stage application with serial number PCT/IN2021/051185, filed on Dec. 17, 2021. The aforementioned PCT international phase application claims the priority from the Indian Provisional Patent Application (PPA) with serial number 202041055581 filed on Dec. 18, 2020, with the title “SYNTHETIC COGNITION (SC) INTUITIVE SYSTEM AND METHOD FOR PREDICTING, ANALYSING AND MANAGING COMPLEX ADAPTIVE AND NON-ADAPTIVE SYSTEMS IN ENGINEERED, OR PARTIALLY BIOLOGICAL APPLICATIONS”. The contents of abovementioned Applications are included in entirety as reference herein.

### BACKGROUND

### Technical Field

The embodiments herein are generally related to the field of computational system for data analytics and artificial intelligence or machine learning systems. The embodiments herein are particularly related to a computational system and method for data analytics and predictability in engineered/non-biological or partially biological systems. The embodiments herein are more particularly related to a synthetic cognition (SC)/intuitive machine learning (IML) technology-based system and method for analyzing, predicting, and managing complex systems in engineered/non-biological or partially biological systems and for translating dataspaces into subjective surrogates of biological cognitive phenomenon, as objective substitutes for biological awareness.

### Description of the Related Art

Typically, natural, engineered, or socio-economic constructs are represented as simple or complex clusters of interrelated systems, which evolve into complex adaptive systems. A complex adaptive system is a system in which a perfect understanding of the individual parts does not automatically convey a perfect understanding of the whole system's behavior. In a complex adaptive system, the complete system is more complex than its parts, and more complicated and meaningful than the aggregate of its parts. They are complex, such that the complex adaptive system are dynamic networks of interactions, and their relationships are not limited to aggregations of individual characteristics, such as the behavior of the ensemble is not predicted by the behavior of the components. They are “adaptive” when individual and collective behavior mutate and self-organize corresponding to the change-initiating micro-event or collection of events or non-adaptive systems. The study of adaptive and non-adaptive systems is highly interdisciplinary, blending insights from natural, social, and other sciences, as well as art and music manifesting a complex datascape canvas that allow for heterogeneous agents, phase transition, emergent behavior, and other potential outcomes.

Therefore, such dynamic complex multi-dimensional systems are better understood as (identifiable) territorial transition clusters among a continuum datascape. Multiple categories of economic information that make up the gross domestic product, multiple clusters of disparate activity that characterize a sophisticated manufacturing process, or the cumulative impact of several factors that collectively signal a state of health as wellness or illness ascribed to each individual and linked to changes related to lifestyle, environment or ageing that predate the diseases that ultimately result from it, optimal state of mechanical harmony collectively representing optimal functioning of a multi system machinery etc., are some examples that require a new paradigm of holistic representation. “Synthetic cognition” is conceived as a technology surrogate for the mental process of apprehension of knowledge and understanding through thought, experience, and the senses that creates a dynamic landscape of perception which reflects from the totality of inputs received and organized. In this context, artificial intelligence and machine learning technology represent the process of funneling the use of the dynamic datascape through differential filtering, selection, and prioritization to enable an ascribed quality of synthetic cognition, economy of mental/computational effort, focus and automatism necessary for rapid and smooth involuntary and voluntary performance.

Therefore, the aspect of managing complexity and surfeit of cognitive data is called “adaptive ignorance” by Alexandra Horowitz, a cognitive scientist. Imagination as the tool of cognition, works in art, science, and literature to exploit and expand the entire cognitive datascape permitting powerful disruptions that distinguish creative and intuitive ideas. Powerful imagination deploys tools beyond logic, purposed or committed neural networks to narrow data not only to the particular and specific, but also extract from its dynamic topography framework for reorganization of data into predictability as intuition, patterns as seeds for ideation. Therefore, expansion of machine learning capability beyond artificial intelligence algorithms requires creation and capacity for investigation of large heterogeneous continuum datascapes representing multiple contiguous phenomena.

Hence, there is need for a method and a system for translating diverse data into datascapes and mechanisms for its organization for the study of complex phenomena and to transform data into small or larger “observable phenomenon”. Furthermore, there is a need of a system to unravel random and non-random behaviour functioning as a whole in varied combinations.

Moreover, there is a need for a novel Synthetic-Cognition to transform static/dynamic/continuum data, into “observable data-portraits” to track the trend of change of individual parameters, trend of change of relational attributes in both the ascribed and unascribed category, and also to compare between similarly sourced continuum datascapes, for predictive modelling and causality implications, integrally in computational systems. Still further there is a need for a system and method able to build data aggregates into interconnected passive or dynamic constructs as building units for creation of 2D/3D morphology and/or synthetic cognition datascapes/data-portraits for enabling delineation of territorial distinctions. Yet there is a need for a system and method for translating datascapes/data-portraits into subjective surrogates of biological cognitive phenomenon, as objective substitutes for biological awareness in robotics, computer-brain interactions and similar applications to acquire/derive auto-recognizable self and relational definable identity, continuum auto-cognition of vulnerability as a measure of perceived risk computed as a ratio of resource availability (such as power etc.,) to response capability and selective “data/axon-gating”. Yet there is a need for a synthetic cognition-based system and method for analysing, predicting, and managing complex adaptive and non-adaptive systems in engineered/nonbiological or partially biological systems and for translating dataspaces into subjective surrogates of biological cognitive phenomenon, as objective substitutes for biological awareness. Yet there is a need for a supplemental computational system and method for data analytics applicable to conventional synthetic cognition, artificial intelligence, machine learning and reinforcement learning techniques.

The abovementioned shortcomings, disadvantages and problems are addressed herein, which will be understood by reading and studying the following specification.

### Objectives of the Embodiments Herein

The primary object of the embodiments herein is to provide a method and system to develop a synthetic cognitive language to represent static/dynamic/continuum data, as an observable data-scape/data-portrait phenomena integrable into computational systems.

Another object of the embodiments herein is to provide a method and a system configured to build data aggregates into interconnected passive or dynamic constructs as building units such as D-cells for creation of two-dimensional (2D)/three-dimensional (3D) morphology architecture and/or synthetic cognition datascapes enabling delineation of territorial distinctions.

Yet another object of the embodiments herein is to provide a method and system to analyse the dynamic relationship between the D-cells arranged in a 2D or 3D morphology, which enables us to delineate the changes in data occurring together structurally or functionally.

Yet another object of the embodiments herein is to provide a method and a system to translate multiple data aggregations into an observable format that provides insights into the working of the entire system without the need to understand its smaller units of function. Holistic/aggregate representation of complex multifaceted events when made into the observable format provide new insights into potential outcomes and also 1× of smaller parts for example, study of a phenomenon such as a tornado not only provides valuable information about known contributing components such as wind speed, but also provides new unanticipated data such as geography and the like (Example: shape, architecture, state of evolution of tornado form, etc.)

Yet another object of the embodiments herein is to provide a method and system to transform diverse heterogenous data or multiple data aggregations into an observable composite/holistic/comprehensive form that is mined or analyzed by several methods such as topology, artificial visualization, and optical/pixel-based analytics without deconstruction into smaller parts but as a whole, the multiple data aggregations. The observable composite/holistic form enables a comprehensive, aggregate functioning of the “whole” of a complex adaptive and non-adaptive systems are accessible to new modes of analysis.

Yet another object of the embodiments herein is to provide a method and system for a novel Synthetic-Cognition to transform static/dynamic/continuum data, into “observable datascapes/data-portraits, (wherein the “continuum” data collected from a machine, or biology as the “datascape” and the “static” snip slice of the continuum datascape pulled out at any chosen time or times is known as the STATIC “DATA-PORTRAIT”. By collecting hourly data-portraits from a 24 HOUR CONTINUIUM DATA-SCAPE) the system and method enables to track (1) a trend of change of individual parameters; (2) a trend of change of relational attributes in both the ascribed and unascribed category; and also, to provide (3) a comparison between similarly sourced continuum datascapes/data-portraits; (4) predictive modelling; and (5) Causality implications in some individual or particular cases. (This is remarkably similar to individual photo portraits taken at different periods in an individual life to demonstrate long and short-term impact of the hardships, suffering, joys, and frustrations etched on to the face in a portrait during a scanning of a photo album)

Yet there is a need for a system and method for translating datascapes/data-portraits into subjective surrogates of biological cognitive phenomenon, as objective substitutes for biological awareness in robotics, computer-brain interactions and similar applications, to define a workable surrogate cognition process using Artificial intelligence to enable the non-biological systems to acquire/derive an auto-recognizable self and relational definable identity, a continuous auto-cognition of vulnerability as a measure of perceived risk computed as a ratio between resource availability (such as a power etc.,) to perception of response capability; and selective “data/axon-gating” capability (which is a Cognitive AI implementable technology to prioritize selective variable access to decision-computation processes to match immediate response needs)

Yet there is a need for a system and method for translating datascapes/data-portraits into subjective surrogates of biological cognitive phenomenon, more optimally and economically by the adaptation of data-scape-data-portrait layering in AI/ML exactly (as being done in the human brain, to see a slice from a short- or long-term exposure to others of data-portrait to decide our psychosocial affinity).

Yet another object of the embodiments herein is to provide a method and system to establish new ways of identifying broad categories of changes such as wellness and illness, harmony, and disharmony, best, better, good, bad, worse, and worst changes that are manifested from observing data as a holistic interconnection of a complex dynamic adaptive system. The method of identifying broad categories of changes might not involve significant alterations in its individual parts but is manifest in its whole, cumulative, or aggregate impact by evaluation of the “whole” as cognitive perception. This enables new possibilities for surveillance, predictability and developing testable models of “causality.”

Yet another object of the embodiments herein is to provide a method and a system to transform small data cohorts, big data, and large amorphous data congregations into an “observable phenomenon” which retains dynamics/signature of known contextual relationship and integrated within a larger contiguous datascape or as part of a heterogeneous composite functional system, as “observable” but unidentified or un-ascribed discoveries. Observability creates opportunity for reverse engineering/modelling/manipulating the composition and character of datascapes intentionally directed observable changes representing “causality” and possessing potentially knowable or intuitive surrogates of “predictability” and “actuality.”

Yet another object of the embodiments herein is to provide a method and a system to translate datascapes into subjective surrogates of biological cognitive phenomenon, as objective substitutes for biological awareness.

Yet another object of the embodiments herein is to enable synthetic cognition as tool for diagnosability and predictability in engineered/non-biological or partially biological systems.

Yet another object of the embodiments herein is to provide an alternative or supplemental “observational/topological/visual analytics” approach in a computational system for data analytics.

Yet another object of the embodiments herein is to provide a system for using a synthetic cognitive language for capturing data and establishing a methodology of collating the captured data in ways that seamlessly integrate diverse, heterogeneous data into a dynamic matrix that helps capture holistic workings of complex dynamic-adaptive functions as a datascape accessible for mining and iteration by conventional and also new artificial intelligence and machine learning approaches.

Yet another object of the embodiments herein is to provide a method and a system to capture broad holistic outcomes in clearly identifiable broad categories such as wellness and illness or harmony or disharmony in biological or non-biological systems with accessible to a reverse approach to iteration and/or manipulation without necessarily having to divide into small constituent components for detailed analysis.

Yet another object of the embodiments herein is to enable mapping of big data as dynamic interconnected large datascapes demonstrating in real time values and characteristics captured by statistical and/or other modes of analysis and functioning as a holistic model for displaying predictability and testing models of causality.

Yet another object of the embodiments herein is to divide a function into its parts, for juxtaposing with other functions (with or without a known relationship, proximate or distant) in various combinations to manifest/predict potential outcomes.

Yet another object of the embodiments herein is to provide a structure with different pathways of progression, wherein the structure provides different functions, by juxtaposing the parts of the functions with different functions will manifest different pathways of progression and gives potential to predict outcome through not just one pathway but all possible pathways.

Yet another object of the embodiments herein is to capture a more natural understanding of natural distribution, interrelation, characteristics, and architecture of the natural constitutive order inherent in complex systems and enable demonstration of alternatives, instead as a combination of distinct separate elements.

Yet another object of the embodiments herein is to arrange the data procured in a nodal architecture and analyze the visual representation of the processed data, which provides the natural distribution and progression of the phenomenon.

Yet another object of the embodiments herein is to translate datascape characteristics using synthetic cognition, usable as tool for diagnosis and prediction in engineered/non-biological or partially biological systems and provide an alternative/supplemental computational approach for adaptive behavior constructs.

Yet another object of the embodiments herein is to provide a method and a system to capture a) holistic workings of complex dynamic-adaptive functions as a datascape accessible for mining and iteration by conventional and also new artificial intelligence and machine learning approaches, b) capture broad outcomes in clearly identifiable categories, and c) to study the evolution of large complex adaptive organizations as a whole.

Yet another object of the embodiments herein is to analyse the signature patterns detected from the conventional or new machine learning algorithms in order to identify the outcomes of the analysis of the complex adaptive systems into identifiable categories like wellness and illness, harmony, and disharmony, best, better, good, bad, worse, and worst outcomes.

Yet another object of the embodiments herein is to provide a method and a system to generate a synthetic-cognition language for capturing data and representing it into computational systems known in the art.

Yet another object of the embodiments herein is to map the change manifestations in the portrait-based phenomenon representation to changes in manifestations of colors, strokes, harmony, form, co-ordination, and other attributes of various forms of art.

Yet another object of the embodiments herein is to provide a method and a system to capture working of complex dynamic adaptive functions for data mining/extraction by artificial intelligence and machine learning approaches.

Yet another object of the embodiments herein is to provide a method and a system that studies the evolution of large complex adaptive organization as a whole with the ability to divide a function into its parts for comparing with other functions in various combinations to predict potential outcome.

Yet another object of the embodiments herein is to provide a method and a system that predicts potential outcomes by dividing a function into its parts to be able to juxtapose with other functions in various combinations.

Yet another object of the embodiments herein is to provide a system and a method to capture a natural understanding complex system instead as a combination of distinct separate elements.

Yet another object of the embodiments herein is to provide a system for organizing the observable data into an architecture that results in natural progression towards organization both as a “new phenomenon” in addition to those deducible by knowledge of parts for example, blood pressure, heart rate and the like.

These and other objects and advantages of the embodiments herein will become readily apparent from the following detailed description taken in conjunction with the accompanying drawings.

### SUMMARY

The following details present a simplified summary of the embodiments herein to provide a basic understanding of the several aspects of the embodiments herein. This summary is not an extensive overview of the embodiments herein. It is not intended to identify key/critical elements of the embodiments herein or to delineate the scope of the embodiments herein. Its sole purpose is to present the concepts of the embodiments herein in a simplified form as a prelude to the more detailed description that is presented later.

The other objects and advantages of the embodiments herein will become readily apparent from the following description taken in conjunction with the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

The various embodiments of the present invention provide a system and a method for synthetic cognition or intuitive machine learning that captures data and collates the captured data in ways that seamlessly integrate diverse, heterogeneous data into a dynamic matrix using a cognitive language, so as to capture holistic workings of complex dynamic-adaptive functions as a datascape accessible for mining and iteration by conventional and also new artificial intelligence and machine learning approaches. The method provided transforms diverse heterogenous data or multiple data aggregations into an observable composite/holistic form that can be mined or analysed by a number of methods such as topology, artificial visualization, and optical/pixel-based analytics without deconstruction into smaller parts but as a whole, the multiple data aggregations. The observable composite/holistic form enables a comprehensive, aggregate functioning of the “whole” of a complex adaptive and non-adaptive systems to be made accessible to new modes of analysis. The new modes of analysis could include any modality that analyses an observable phenomenon. The embodiments of the system and method provide the options of taking out as smaller units as data-portraits which are analysed in relation to the study of conventional “smaller parts” analysis

According to one embodiment herein, a system for managing complex adaptive and non-adaptive systems using a novel language and system enabling synthetic cognition/intuitive machine learning is provided. In an embodiment the system is configured to capture broad outcomes in clearly identifiable categories such as health and illness or harmony or disharmony in systems with a reverse approach to iteration without having to divide into small components for detailed analysis. The system is configured to divide a function into its parts to be able to juxtapose with other functions in various combinations to predict potential outcomes. The system captures a natural understanding of complex systems instead as a combination of distinct separate elements. The system enables synthetic cognition to be used as tool for diagnosis and prediction in engineered/non-biological or partially biological systems and provides an alternative computational system for data analytics.

According to one embodiment herein, a method for managing complex adaptive and non-adaptive systems for synthetic cognition/intuitive machine learning is provided. In an embodiment, the method includes capturing a broad outcome in clearly identifiable categories in systems with a reverse approach to iteration without having to divide into small components for detailed analysis. The method also includes dividing a function into its parts to be able to juxtapose with other functions in various combinations to predict potential outcomes. The method also includes generating a synthetic language representation of static/dynamic/continuum data, to be integrated into computational systems using a synthetic cognition language. The method also includes capturing a natural understanding of complex systems as a combination of distinct separate element.

The various embodiments of the present invention provide a system architecture comprising plurality of modules responsible for managing complex adaptive system using synthetic cognition or intuitive machine learning approach, wherein the plurality of modules includes data collection module, data processing module, grid generation module and data mining module. The data collection module captures and collates the plurality of data and then routes the captured plurality of data into the data processing module. The plurality of data is of the type asynchronous and comprises of small data, big data, and large amorphous data congregation. The data processing module cleans up the plurality of asynchronous data and converts the asynchronous data into synchronous data, further encodes the synchronous data and finally normalize the synchronous data qualitatively as well as quantitatively. Further the normalized synchronous data is seamlessly integrated as dynamic matrix into a grid generation module using a synthetic cognitive language so as to capture holistic working of complex dynamic-adaptive functions such as a datascape. The grid generation module performs categorization of data in nodal architecture by classifying the data into four clusters such as zone of direct active, zone of potentiality, zone of probability and zone of enquiry. Furthermore, the grid generation module converts the categorized data in nodal architecture into interconnected passive or dynamic constructs such as building units for the creation of two-dimensional (2D)/three-dimensional (3D) morphology architecture and/or synthetic cognition datascapes enabling delineation of territorial distinctions by means of grid conversion algorithm. Further the 2D/3D morphology and datascapes from the grid generation module is passed to the data mining module, wherein the data mining module helps to assess or visualize the grid generated datascapes and 2D/3D morphology, by analyzing or detecting the signature patterns of datascapes through a signature pattern detection (algorithm?) of the data mining module. Further, the data mining module helps in mining and iteration of the datascapes by conventional and also NEW artificial intelligence and machine learning approaches.

According to one embodiment herein, a method for predicting, analyzing and managing, complex adaptive and non-adaptive system by means of synthetic cognition or intuitive machine learning comprising the steps: capturing and collating plurality of data, wherein the plurality of data is of the type asynchronous and comprises of small data, big data and large amorphous data congregation; cleaning up the obtained plurality of data and converting asynchronous type of plurality of data into synchronous data followed by encoding the synchronous data and normalizing the synchronous data qualitatively as well as quantitatively. Categorizing the normalized synchronous data in a nodal architecture and converting the categorized synchronous data into interconnected passive or dynamic constructs such as building units for the creation of two-dimensional (2D)/three-dimensional (3D) morphology architecture and/or synthetic cognition datascapes enabling delineation of territorial distinctions by means of grid conversion algorithm. Furthermore, detecting the signature pattern of 2D/3D morphology architecture and/or synthetic cognition datascapes by means of signature pattern detection, wherein the signature pattern detection helps in visualizing the observable datascapes or observable phenomenon of the 2D/3D morphology architecture and/or synthetic cognition datascapes, wherein the observable phenomenon retains the dynamics/signature of known contextual relationship and provides insights into the working of the whole system without the need to have understanding of its smaller units of function. Holistic/aggregate representation of complex multifaceted events when made into the observable format provide new insights into potential outcomes and also behavior of smaller parts. Further, mining and iterating the observable phenomenon/holistic form by conventional and also NEW artificial intelligence and machine learning approaches without deconstruction into smaller parts but as a whole, observable phenomenon.

Therefore, the embodiments herein provides a method and system that translates small or large data to be made available as an observable phenomena where instead of individual elements that make up various components of an activity or function is viewed in its totality and studied independent of reference to its constituent parts as various attributes of a phenomenon, thereby enabling creation of broad categories such as, wellness and illness or as simply ascribed new categories which are only observable and exist in the totality of a phenomenon but might not necessarily represent an identifiable attribute when broken into its parts.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should, and arc intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

### BRIEF DESCRIPTION OF THE DRAWINGS

The other objects, features and advantages will occur to those skilled in the art from the following description of the preferred embodiment and the accompanying drawings in which:

FIG. 1 illustrates a system architecture comprising plurality of modules responsible for managing complex adaptive systems using synthetic cognition/intuitive machine learning, according to an embodiment herein.

FIG. 1A-1F illustrates an example application scenario of the system architecture relating to the present technology for analysis of a fluid in a soft pipe, according to an embodiment herein.

FIG. 2 illustrates a process flowchart on stepwise process involved in predicting, analyzing, and managing complex adaptive and non-adaptive system in engineered/non-biological, or partially biological applications, according to an embodiment herein.

FIG. 2A-2C illustrates the stepwise manner details on the formation of D-CELL and D-TISSUE based on geometric organization of D-AXONS, according to an embodiment herein.

FIG. 2D-2G depict a geometric organization of D-AXONs of the present system in a closed or a series configuration, according to an embodiment herein.

FIG. 2H illustrates a D-CELL comprising centroid vector representing the relational magnitude and direction in accordance with an exemplary scenario, according to an embodiment herein.

FIG. 2I illustrates an addition of new grid line/data along the horizontal or vertical direction, corresponding to an exemplary D-CELL, according to an embodiment herein.

FIG. 2J illustrates a graphical representation of horizontal and vertical components of the centroid along with the gridline variables, according to an embodiment herein.

FIG. 2K-2L illustrates the juxtaposing the data in the grid in various combinations, according to an embodiment herein.

FIG. 2M illustrates the escribed and unescribed data relationship, according to an embodiment herein.

FIG. 2N illustrates a D-TISSUE unit, corresponding to an exemplary D-AXON, in accordance with an exemplary scenario, according to an embodiment herein.

FIG. 2O illustrates a time chronology square of the D-TISSUE unit, according to an embodiment herein.

FIG. 3A illustrates a derivative function versus saturation plot indicating an aggregate impact of blood pressure, skin temperature and oxygen saturation, in accordance with an exemplary scenario, according to an embodiment herein.

FIG. 3B illustrates a clustered data and sequentially aligned data as cluster of known maximal conformity, in accordance with an exemplary scenario, according to an embodiment herein.

FIG. 3C illustrates a nodal organization hierarchy, according to an embodiment herein.

FIG. 3D illustrates D-CELL dimension determinants, according to an embodiment herein.

FIG. 3E illustrates a maximal extent of data flux determination, in accordance with an exemplary scenario, according to an embodiment herein.

FIG. 3F-3I illustrates a D-CELL Plasma centroid shift surrogate for net activity, according to an embodiment herein.

FIG. 3J illustrates the process steps involved in development of synthetic cognitive language to represent static/dynamic/continuum data, observable phenomena integrable into computational system, according to an embodiment herein.

FIG. 4A illustrates an exemplary taxonomic organization of an exemplary cell unit, according to an embodiment herein.

FIG. 4B illustrates an exemplary D-CELL where each node represents measurable known functional relationship, according to an embodiment herein.

FIG. 4C illustrates a series of nodes, according to an embodiment herein.

FIG. 4D illustrates a convergent aggregate impact of four datasets, according to an embodiment herein.

FIG. 4E illustrates an aggregate impact of four nodes and eight data sets, according to an embodiment herein.

FIG. 4F illustrates an example D-TISSUE with 4 units of cohorts, according to an embodiment herein.

FIG. 4G illustrates an example D-TISSUE 8 data points 8, and 16 nodes, in accordance with an exemplary scenario, according to an embodiment herein.

FIG. 4H illustrates a linear organization of D-TISSUE, according to an embodiment herein

FIG. 4I illustrates an exemplary categorization of the cohorts as per performance into various categories, according to an embodiment herein.

FIG. 4J illustrates an example of D-CELL stratification resulting in an adaptive tissue system, according to an embodiment herein.

FIG. 4K illustrates an exemplary organization of categories of cohorts according to stage of change, according to an embodiment herein.

FIG. 4L illustrates an exemplary data cluster, according to an embodiment herein.

FIG. 5 illustrates the process steps involved in method to build data aggregates into interconnected passive or dynamic constructs as building units for the creation of 2D/3D morphology architecture and/or synthetic cognition datascapes enabling delineation or territorial distinctions, according to an embodiment herein.

FIG. 6A illustrates the method transform different types of data into “observable phenomenon” that retains dynamic signatures, according to an embodiment herein.

FIG. 6B illustrates the method to capture broad holistic outcomes in clearly identifiable broad categories in biological or non-biological system, according to an embodiment herein.

FIG. 6C illustrates the method for testing “causality” and “predictability”, according to an embodiment herein.

FIG. 7A illustrates the method to transform multiple data aggregations into an “observable phenomenon”, which provides insight into working of entire system, according to an embodiment herein.

FIG. 7B illustrates the method to transform diverse heterogenous data into observable holistic representation, according to an embodiment herein.

FIG. 7C illustrates a topological organization of the datascape, according to an embodiment herein.

FIG. 8 illustrates a method for organizing the observable data into an architecture that results in natural progression, according to an embodiment herein.

FIG. 9 illustrates an exemplary datascape of cyber sensorium, according to an embodiment herein.

Although the specific features of the present invention are shown in some drawings and not in others. This is done for convenience only as each feature may be combined with any or all of the other features in accordance with the present invention.

### DETAILED DESCRIPTION OF THE EMBODIMENTS HEREIN

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that other changes may be made without departing from the scope of the embodiments. The following detailed description is therefore not to be taken in a limiting sense.

The various embodiments of the present invention provide a synthetic cognition (SC)/intuitive machine learning (IML) technology-based system and method for analyzing, predicting, and managing complex systems in engineered/non-biological or partially biological systems. The embodiments herein also provide a method for translating datascapes/data-portraits into subjective surrogates of biological cognitive phenomenon, as objective substitutes for biological awareness.

The various embodiments of the present invention provide a system architecture comprising plurality of modules responsible for managing complex adaptive system using synthetic cognition or intuitive machine learning approach, wherein the plurality of modules includes data collection module, data processing module, grid generation module and data mining module. The data collection module captures and collates the plurality of data and then routes the captured plurality of data into the data processing module. The plurality of data is of the type asynchronous and comprises of small data, big data, and large amorphous data congregation. The data processing module cleans up the plurality of asynchronous data and converts the asynchronous data into synchronous data, further encodes the synchronous data and finally normalize the synchronous data qualitatively as well as quantitatively. Further the normalized synchronous data is seamlessly integrated as dynamic matrix into a grid generation module using a synthetic cognitive language so as to capture holistic working of complex dynamic-adaptive functions such as a datascape. The grid generation module performs categorization of data in nodal architecture by classifying the data into four clusters such as zone of direct active, zone of potentiality, zone of probability and zone of enquiry. Furthermore, the grid generation module converts the categorized data in nodal architecture into interconnected passive or dynamic constructs such as building units for the creation of two-dimensional (2D)/three-dimensional (3D) morphology architecture and/or synthetic cognition datascapes enabling delineation of territorial distinctions by means of grid conversion algorithm. Further the 2D/3D morphology and datascapes from the grid generation module is passed to the data mining module, wherein the data mining module helps to assess or visualize the grid generated datascapes and 2D/3D morphology, by analyzing or detecting the signature patterns of datascapes through a signature pattern detection technique/algorithm of the data mining module. Further, the data mining module helps in mining and iteration of the datascapes by conventional and also a new artificial intelligence and machine learning approaches.

According to one embodiment herein, the synthetic cognition is a technology surrogate for the mental process of apprehension of knowledge and understanding to create a dynamic data landscape reflecting the totality of inputs that is mined for specific information.

According to one embodiment herein, the zone of direct active of nodal architecture comprises of data where there exists deterministic relationship between the elements. For instance, temperature and pressure, there exists a directional relationship between these elements defined by a formula or an equation. The zone of potentiality of nodal architecture comprises of data where there exists a surrogate relationship between the elements. For instance, dynamic friction co-efficient and Reynold's number. There is no direct equation to relate these elements, but it has been found that there exists a linear relationship between them. The zone of probability of nodal architecture comprises of data where there exists probabilistic cause-effect relationship between the elements. For instance, the relationship between viscosity and the cross-sectional area. Though the viscosity and cross-sectional area of the pipe are not connected, the structural progressive changes in the cross-sectional area affect the dynamic changes in viscosity. Furthermore, the zone of enquiry of nodal architecture comprises of data where there exists a possible cause-effect relationship between the elements that are not yet defined by any theorem. For instance, the relationship between the flow rate and concentration of oxygen in the fluid. The relationship is unknown and can only be manifested when the changes are observed together with the other categories.

According to one embodiment herein, the synthetic cognitive language is a representation of the whole phenomenon including all the static, dynamic, and continuum data. The synthetic cognitive language is assigned not only on the basis of known or ascribed relationships or design but also un-ascribed. Furthermore, the synthetic cognition language can also be used to capture data and represent the data into computational systems known in the art.

According to one embodiment herein, the building units for the creation of two-dimensional (2D)/three-dimensional (3D) morphology architecture and/or synthetic cognition datascapes generated via grid generation module includes D-AXONS, D-CELLS and D-TISSUES. The D-AXONS includes data from individual elements converted into a line, where the line width and colour of the data are mapped to disparate attributes of the data. The D-CELL is formed when the D-AXONS are arranged in a grid-like pattern in a nodal architecture. A D-CELL is a holistic data matrix of the system at a single timestamp. Furthermore, the D-TISSUE is formed as D-CELL timestamps are stacked together to form a large data matrix that captures the whole dynamic or a chronological event of a complex adaptive system. Sequentially, various complex adaptive systems stack to form even a larger data matrix of D-ORGAN and D-ORGANISM, respectively. Hence, the dynamic relationship between the cells arranged in a 2D or 3D morphology enables to delineate the changes occurring together in which the data is related structurally or functionally.

According to one embodiment herein, the signature patterns detected by the data mining module including the conventional or new machine learning algorithms such as artificial intelligence and machine learning approaches identify the outcomes of the analysis of the complex adaptive systems into identifiable categories such as wellness and illness, harmony, and disharmony, best, better, good, bad, worse, and worst. Further, while the datascapes are visualized via data mining module the changes due to any structural modifications are manifested first and the functional changes follow the structural manifestations. Hence, the approach eliminates the reliance/reliability only on understanding through a reductionist approach of studying small functional units individually in order to understand the entire system.

According to one embodiment herein, the system architecture is configured to capture broad outcomes in clearly identifiable categories such as health and illness or harmony or disharmony in systems with a reverse approach to iteration without having to divide into small components for detailed analysis. The system facilitates dividing a function into its parts to be able to juxtapose with other functions in various combinations to predict potential outcomes. Hence, the system captures a natural understanding of complex systems instead as a combination of distinct separate elements. Furthermore, the system enables synthetic cognition to be used as tool for diagnosis and prediction in engineered/non-biological or partially biological systems and provides an alternative computational system for data analytics. In addition, the mining and iteration of the datascapes by conventional and also NEW artificial intelligence and machine learning approaches includes pattern recognition, image classification, Generative Adversarial Network, Convolutional Neural Network, and the like.

According to one embodiment herein, a method for predicting, analyzing and managing, complex adaptive and non-adaptive system by means of synthetic cognition or intuitive machine learning comprising the steps: capturing and collating plurality of data, wherein the plurality of data is of the type asynchronous and comprises of small data, big data and large amorphous data congregation; cleaning up the obtained plurality of data and converting asynchronous type of plurality of data into synchronous data followed by encoding the synchronous data and normalizing the synchronous data qualitatively as well as quantitatively. Categorizing the normalized synchronous data in a nodal architecture and converting the categorized synchronous data into interconnected passive or dynamic constructs such as building units for the creation of two-dimensional (2D)/three-dimensional (3D) morphology architecture and/or synthetic cognition datascapes enabling delineation of territorial distinctions by means of grid conversion algorithm. Furthermore, detecting the signature pattern of 2D/3D morphology architecture and/or synthetic cognition datascapes by means of signature pattern detection, wherein the signature pattern detection helps in visualizing the observable datascapes or observable phenomenon of the 2D/3D morphology architecture and/or synthetic cognition datascapes, wherein the observable phenomenon retains the dynamics/signature of known contextual relationship and provides insights into the working of the whole system without the need to have understanding of its smaller units of function. Holistic/aggregate representation of complex multifaceted events when made into the observable format provide new insights into potential outcomes and also behavior of smaller parts. Further, mining and iterating the observable phenomenon/holistic form by conventional and also artificial intelligence and machine learning approaches without deconstruction into smaller parts but as a whole, observable phenomenon.

According to one embodiment herein, the categorizing the normalized synchronous data in a nodal architecture comprises of classifying the data into four clusters such as zone of direct active, zone of potentiality, zone of probability and zone of enquiry. the zone of direct active of nodal architecture comprises of data where there exists deterministic relationship between the elements. For instance, temperature and pressure, there exists a directional relationship between these elements defined by a formula or an equation. The zone of potentiality of nodal architecture comprises of data where there exists a surrogate relationship between the elements. For instance, dynamic friction co-efficient and Reynold's number. There is no direct equation to relate these elements, but it has been found that there exists a linear relationship between them. The zone of probability of nodal architecture comprises of data where there exists probabilistic cause-effect relationship between the elements. For instance, the relationship between viscosity and the cross-sectional area. Though the viscosity and cross-sectional area of the pipe are not connected, the structural progressive changes in the cross-sectional area affect the dynamic changes in viscosity. Furthermore, the zone of enquiry of nodal architecture comprises of data where there exists a possible cause-effect relationship between the elements that are not yet defined by any theorem. For instance, the relationship between the flow rate and concentration of oxygen in the fluid. The relationship is unknown and manifested when the changes are observed together with the other categories. Hence the data when arranged in a nodal architecture and the visual representation of the processed data represents the natural distribution and progression of the phenomenon. The categorization of synchronous data in nodal architecture helps to capture a more natural understanding of natural distribution, interrelation, characteristics, and architecture of the natural constitutive order inherent in complex systems and enable demonstration of alternatives, instead of as a combination of distinct separate elements.

According to one embodiment herein, the interconnected passive or dynamic constructs such as building units for the creation of two-dimensional (2D)/three-dimensional (3D) morphology architecture and/or synthetic cognition datascapes includes D-AXONS, D-CELLS and D-TISSUES. The D-AXONS includes data from individual elements converted into a line, where the line width and colour of the data are mapped to disparate attributes of the data. The D—CELL is formed when the D-AXONS are arranged in a grid-like pattern in a nodal architecture. A D-CELL is a holistic data matrix of the system at a single timestamp. Furthermore, the D—TISSUE is formed as D-CELL timestamps are stacked together to form a large data matrix that captures the whole dynamic or a chronological event of a complex adaptive system. Sequentially, various complex adaptive systems stack to form even a larger data matrix of D-ORGAN and D-ORGANISM, respectively. Hence, the dynamic relationship between the cells arranged in a 2D or 3D morphology enables to delineate the changes occurring together in which the data is related structurally or functionally.

According to one embodiment herein, the method for predicting, analyzing, and managing, complex adaptive and non-adaptive system by means of synthetic cognition or intuitive machine learning helps in dividing a function into its parts to be able to juxtapose with other functions (with or without a known relationship, proximate or distant) in various combinations to predict potential outcomes. The elements of the 2D/3D morphology datascapes D-AXON of a function of D-CELL can be arranged or juxtapose in different variations by changing the position of the D-AXON in the D-CELL. Similarly, different permutations and combinations of D-CELL arrangements can also be analysed to identify structural and functional variants of the complex adaptive system. Hence, the same structure with different pathways of progression will provide different functions. Therefore, by juxtaposing the parts of the functions with different functions helps to manifest different pathways of progression and gives potential to predict outcome through not just one pathway but all possible pathways.

According to one embodiment herein, the datascapes/data-portraits are translated into subjective surrogates of biological cognitive phenomenon, as objective substitutes for biological awareness. Furthermore, the changes in the subject surrogates of a cognitive phenomenon are observed as a subthreshold change in the system before the system reaches a critical threshold. Hence, these changes are often observed as a territorial shift in one or more of the parts of the system and their associated dynamic signature progresses towards the critical threshold signature. Therefore, by tracking the territorial shift and the dynamic signature of these territories, the subjective surrogate changes can be translated as objective biological awareness. In addition, mapping of big data as dynamic interconnected large datascapes demonstrates the real-time values and characteristics captured by statistical and/or other modes of analysis and functioning as a holistic model for displaying predictability and testing models of causality. Furthermore, the datascape characteristics as synthetic cognition, can be used as a tool for diagnosis and prediction in engineered/non-biological or partially biological systems and provide an alternative/supplemental computational approach for adaptive behaviour constructs. Hence, the data when represented as a whole can be understood both functionally and structurally.

According to one embodiment herein, the signature pattern helps to identify the outcomes of the analysis of the complex adaptive systems into identifiable categories such as wellness and illness, harmony, and disharmony, best, better, good, bad, worse, and worst. Hence the method enables to capture broad holistic outcomes in clearly identifiable broad categories such as wellness and illness or harmony or disharmony in biological or nonbiological systems with accessible to a reverse approach to iteration and/or manipulation without necessarily having to divide into small constituent components for detailed analysis. Furthermore, the broad categories of change manifest the system as a whole and does not involve significant alterations in its individual parts, with respect to enablement of new possibilities for surveillance, predictability, and developing testable models of “causality.” The broad and clearly identifiable categories of change such as wellness and illness, harmony, and disharmony, best, better, good, bad, worse, and worst are manifested from observing data as a holistic interconnection of a complex dynamic adaptive system. The changes in the data when represented individually are predictable only when the data crosses its threshold of detection. Hence, when the data is observed as a whole, the changes due to any structural modifications are manifested first and the functional changes follow the structural manifestations. Thus, enabling the new possibilities of surveillance, predictability, and causal model.

According to one embodiment herein, the observable phenomenon provides insight into working of the entire system without the need to understand its smaller units of function. The visual representation of the observable phenomenon enables the manifestation of broad categories of changes. Therefore, when the data is observed as a visual representation of the observable phenomenon, the changes due to any structural modifications are manifested first and the functional changes follow these manifestations. Hence, this eliminates the need to understand the small functional units individually in order to understand the entire system. Furthermore, the observable phenomenon retains the dynamic/signature of known contextual relationships and is also integrated within a larger contiguous datascape or as a part of a heterogenous composite functional system as “observable” but unidentified or un-ascribed discoveries. The observable but unidentified or un-ascribed discoveries are detected by signature patterns and the progression pathways of observable phenomenon is detected earlier even before the endpoint is observed. In addition, the observable phenomenon is organized into an architecture that results in the natural progression towards organization both as a “new phenomenon” in addition to those deducible by knowledge of parts for example, blood pressure, heart rate, and the like. The approach on the system by deducing the system into known parts will not give a complete understanding of the system or the phenomenon. The different pathways of progression lead to the same endpoint. Hence, by deducing the knowledge to its parts can help understand the endpoint but not the cause. Therefore, the holistic/comprehensive/integrated approach helps to understand the natural progression of the phenomenon.

According to one embodiment herein, the observability of the phenomenon creates opportunity for reverse engineering/modelling/manipulating the composition and character of datascapes intentionally directed observable changes representing “causality” and possessing “potentially knowable or intuitive” surrogates of predictability and actuality.

According to one embodiment herein, during mining and iterating the observable phenomenon/holistic form by conventional and also artificial intelligence and machine learning approaches the data is observed as a visual representation of the phenomenon and the changes due to any structural modifications are manifested first and the functional changes follow the structural manifestations. Hence, this eliminates reliance only on understanding through a reductionist approach of studying small functional units individually in order to understand the entire system. Therefore, data when converted into visual representation is mined or iterated by conventional machine learning approaches such as pattern recognition, image classification, Generative Adversarial Network and Convolutional Neural Network.

According to one embodiment herein, the observable phenomenon in its holistic/integrated form is mined or analyzed by a number of methods such as topology, artificial visualization, and optical/pixel-based analytics without deconstruction into smaller parts but as a whole, the multiple data aggregations. The data when converted into an observable visual medium, various topographical analysis is performed to understand the territorial changes, artificial visualization, and pixel analytics, which helps to capture the dynamic change in the signatures due to structural or functional changes in the complex adaptive system.

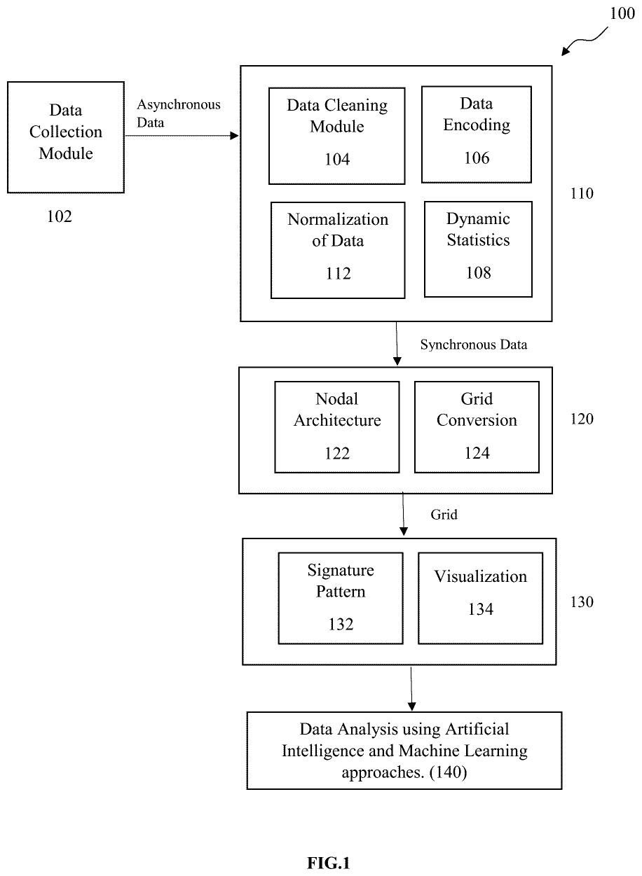

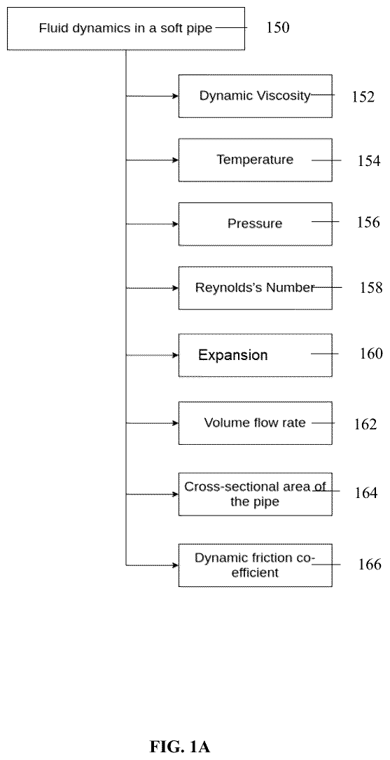

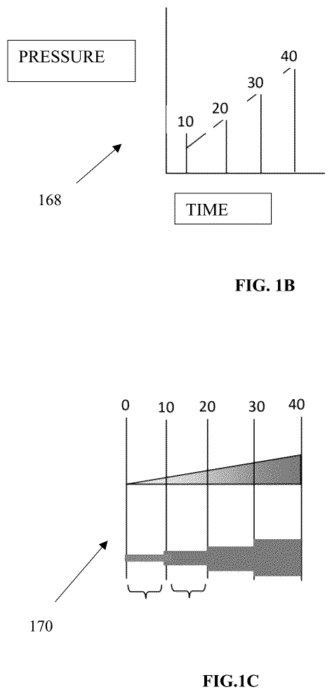

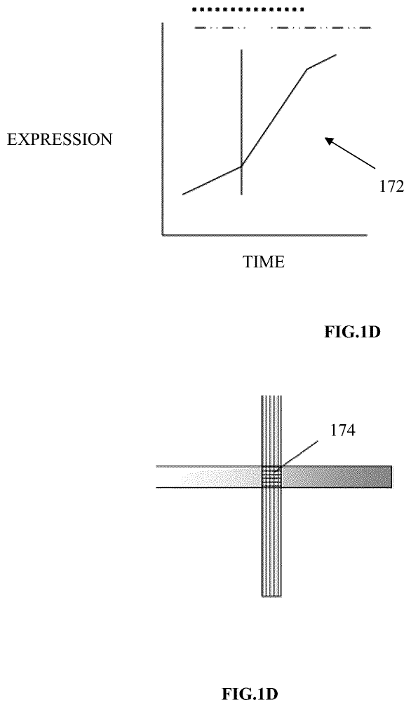

According to one embodiment herein, FIG. 1 illustrates a system architecture comprising plurality of modules responsible for managing complex adaptive systems using synthetic cognition/intuitive machine learning. FIG. 1 illustrates a system architecture 100 comprising plurality of modules such as data collection module 102, data processing module 110, grid generation module 120 and data mining module 130 responsible for managing complex adaptive system using synthetic cognition or intuitive machine learning approach. The data collection module 102 captures and collates the plurality of data and then routes the captured plurality of data into the data processing module 110. The plurality of data is of the type asynchronous and comprises of small data, big data, and large amorphous data congregation. Further, the data processing module 110 cleans up the plurality of data by means of data cleaning module 104 and converts asynchronous data into synchronous data. The data processing module 110 then encodes the synchronous data by means of data encoding module 106, analyses the dynamic statistics 108 of the synchronous data and finally normalizes the synchronous data qualitatively as well as quantitatively by means of normalization module 112. Furthermore, the normalized synchronous data from the data processing module 110 is seamlessly integrated as dynamic matrix into a grid generation module 120 by means of a synthetic cognitive language so as to capture holistic working of complex dynamic-adaptive functions such as a datascape. The grid generation module 120 performs categorization of data in nodal architecture 122 by classifying the data into four clusters such as zone of direct active, zone of potentiality, zone of probability and zone of enquiry. Furthermore, the grid generation module 120 converts the categorized data in nodal architecture 122 into interconnected passive or dynamic constructs such as building units for the creation of two-dimensional (2D)/three-dimensional (3D) morphology architecture and/or synthetic cognition datascapes enabling delineation of territorial distinctions by means of grid conversion algorithm 124. The 2D/3D morphology and datascapes from the grid generation module 120 is passed to the data mining module 130, wherein the data mining module 130 helps to assess or visualize 134 the grid generated datascapes and 2D/3D morphology, by analyzing or detecting the signature patterns of datascapes through a signature pattern detection 132 of the data mining module 130. Moreover, the data mining module 130 helps in mining and iteration of the datascapes by conventional and also artificial intelligence and machine learning approaches 140.

According to one embodiment herein, FIG. 1A-1F illustrates an example application scenario of the system architecture relating to the present technology for analysis of a fluid in a soft pipe. FIG. 1A illustrates various parameters associated with a fluid dynamic in a soft pipe 150, such as dynamic viscosity 152, Temperature 154, Pressure 156, Reynold's number 158, expansion 160, volume flow rate 162, cross-sectional area of pipe 164 and dynamic friction co-efficient 166. FIG. 1B illustrates a pressure (Y-axis) versus time (X-axis) curve 168. FIG. 1C illustrates an intermittent data acquisition 170 and FIG. 1D illustrates an expansion (Y-axis) versus time (X-axis) curve 172. FIG. 1E illustrates a D-NODE 174 corresponding to the example application scenario of FIGS. 1A-1D. The D-NODE 174 visualizes and indicates aggregate of two data sets in terms of node dimension, node chromatic shift, and node intensity/opacity.

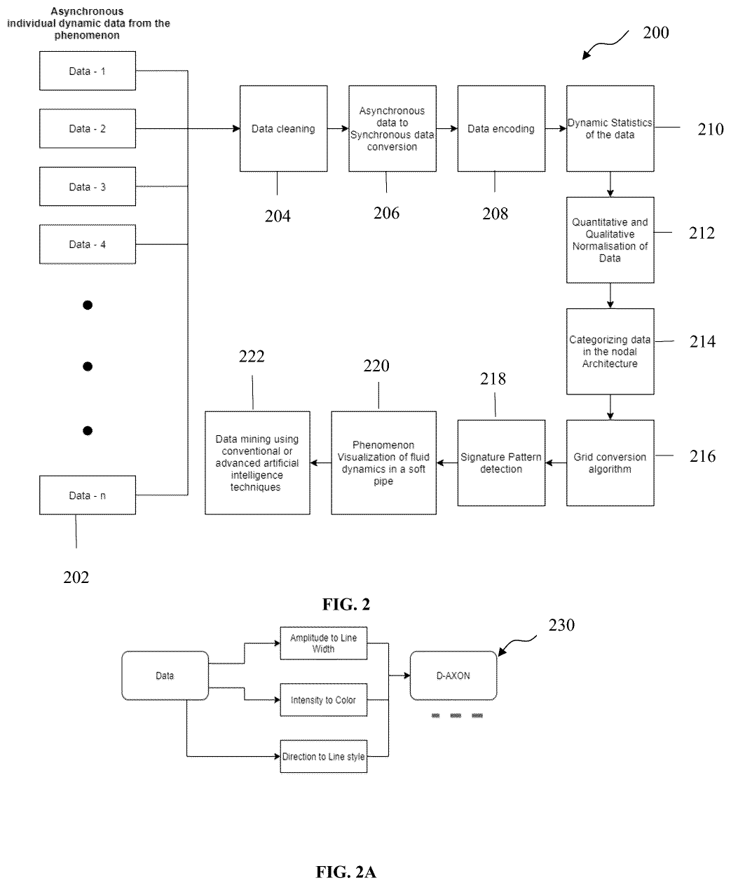

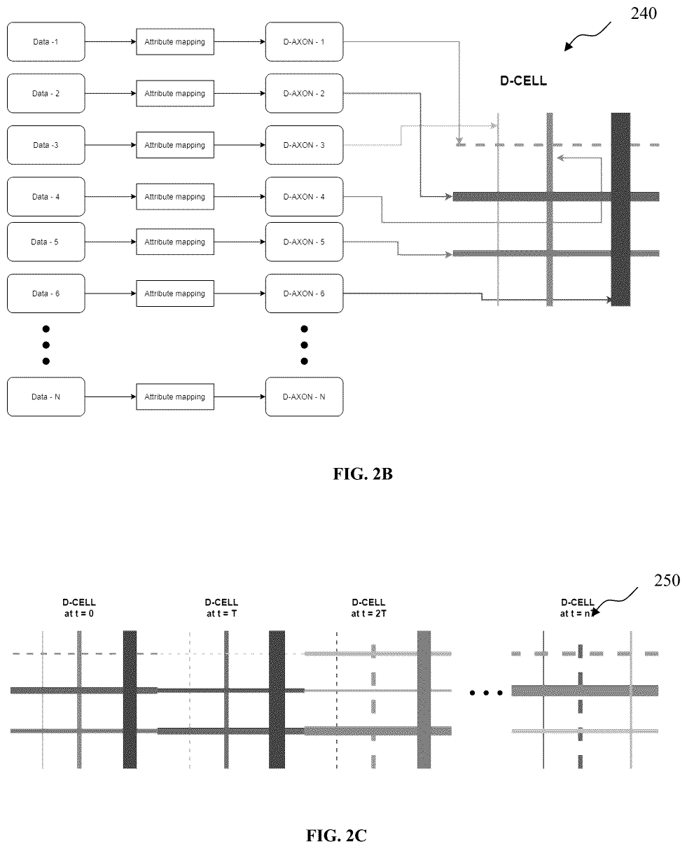

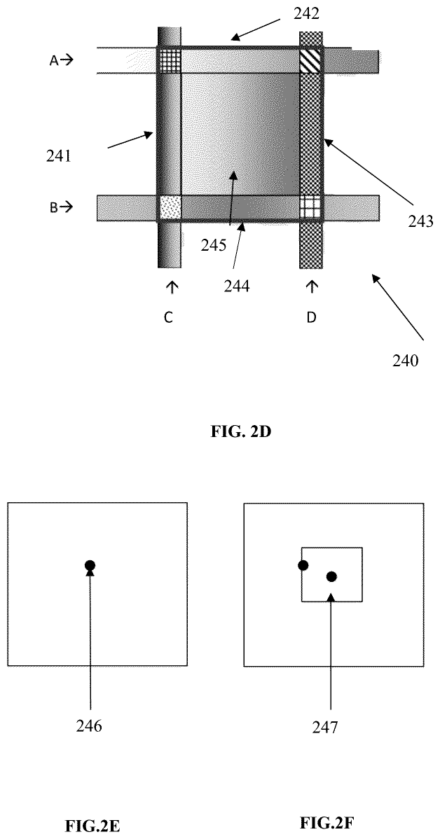

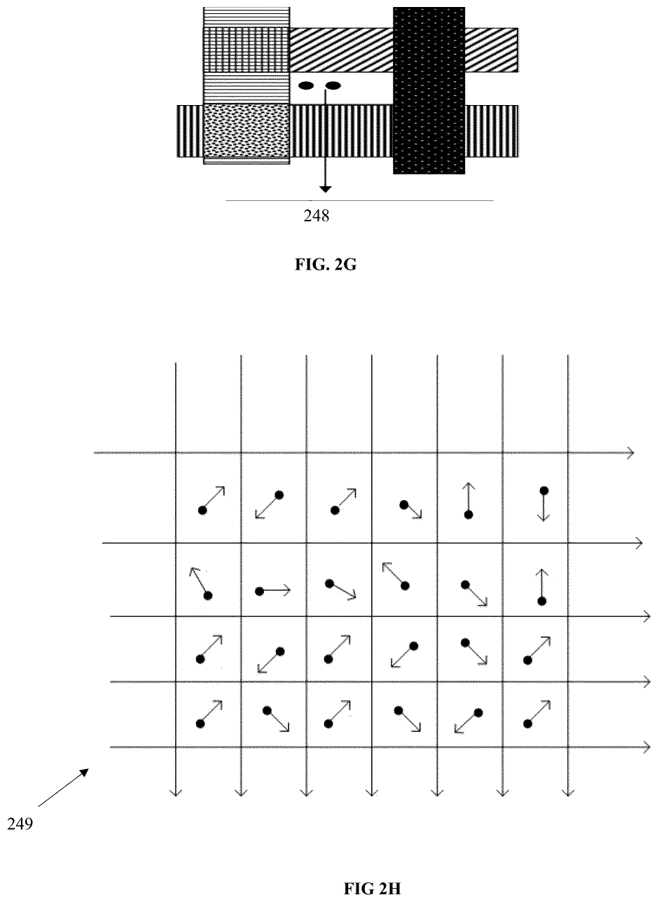

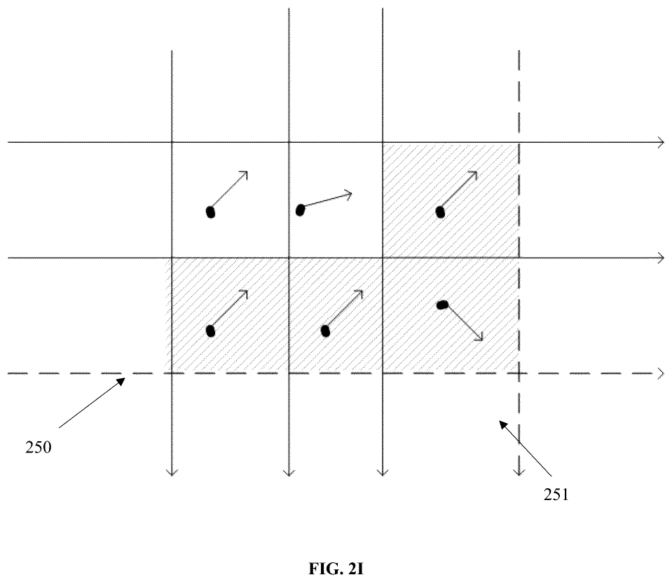

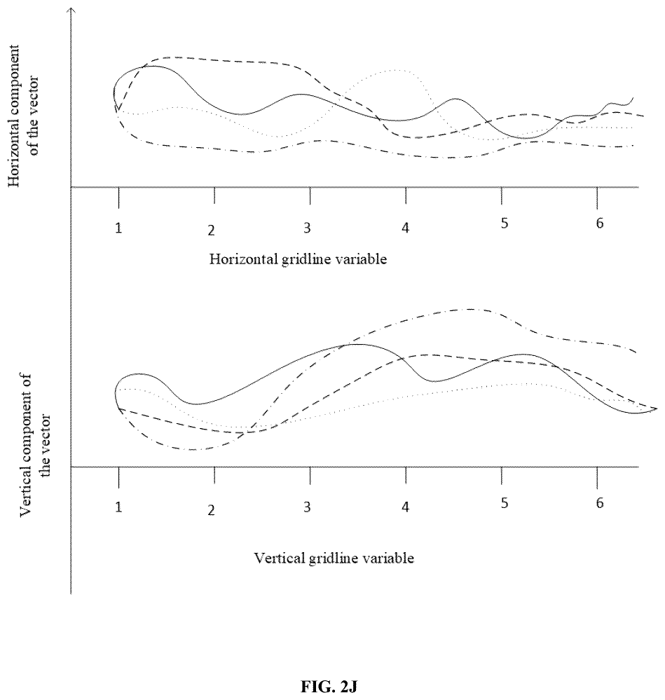

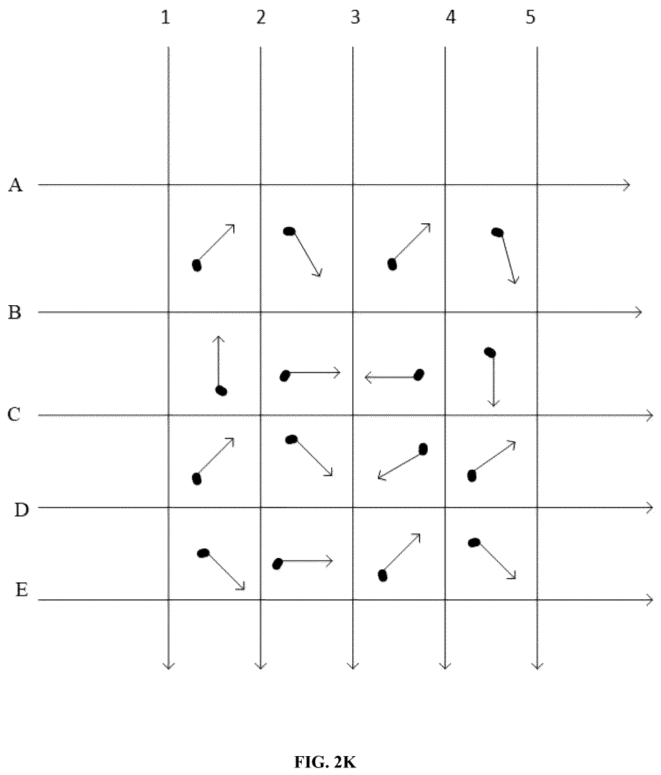

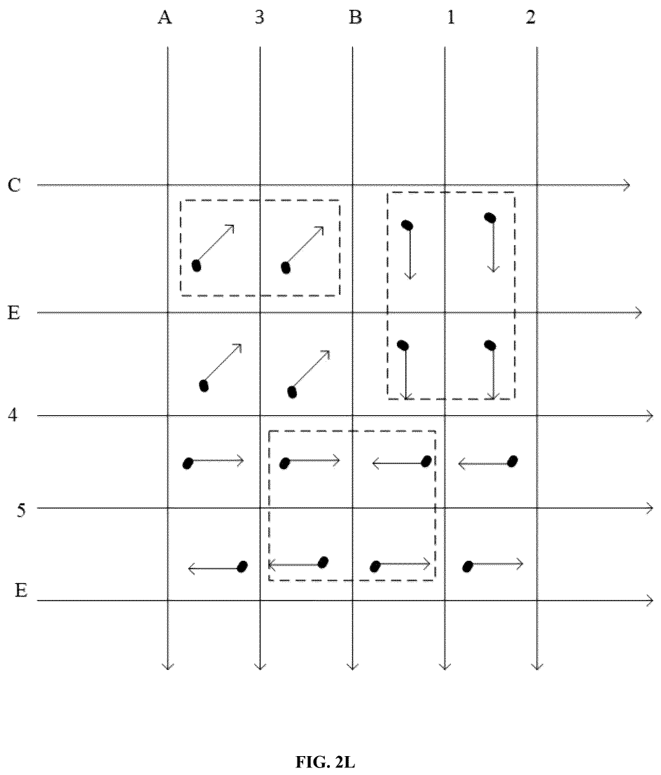

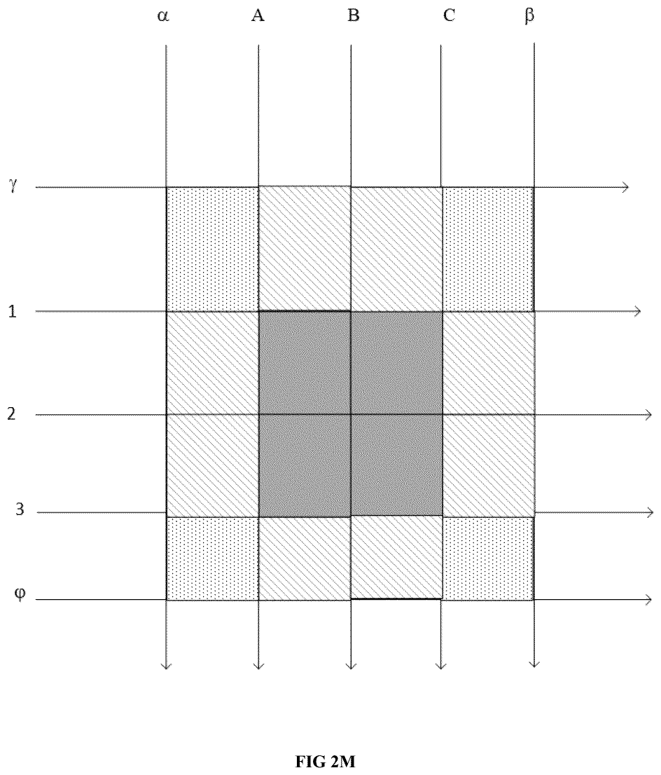

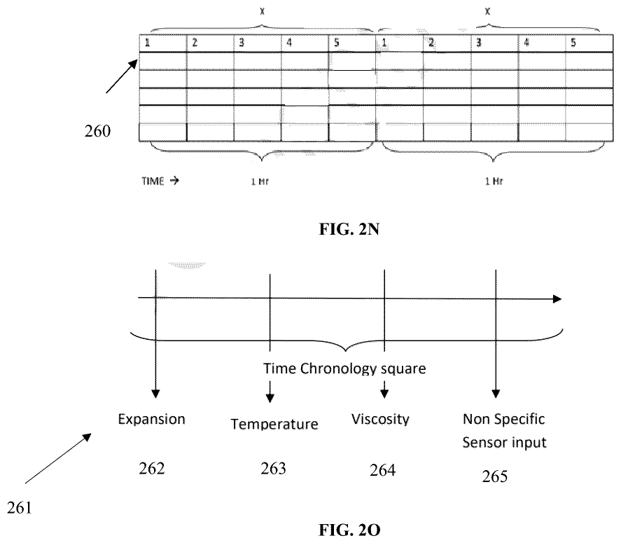

According to one embodiment herein, FIG. 2 illustrates a process flowchart on stepwise process involved in predicting, analyzing, and managing complex adaptive and non-adaptive system in engineered/non-biological, or partially biological applications. FIG. 2 illustrates a flow chart 200 a method for predicting, analyzing, and managing, complex adaptive and non-adaptive system by means of synthetic cognition or intuitive machine learning comprising the steps: capturing and collating plurality of data 202, wherein the plurality of data is of the type asynchronous and comprises of small data, big data, and large amorphous data congregation. Followed by cleaning up the obtained plurality of data 204 and converting asynchronous type of plurality of data into synchronous data 206 then encoding the synchronous data 208, analyzing the dynamic statistics of the synchronous data 210 and normalizing the synchronous data qualitatively as well as quantitatively 212. Furthermore, the method consists of Categorizing the normalized synchronous data in a nodal architecture 214 and converting the categorized synchronous data into interconnected passive or dynamic constructs such as building units for the creation of two-dimensional (2D)/three-dimensional (3D) morphology architecture and/or synthetic cognition datascapes enabling delineation of territorial distinctions by means of grid conversion algorithm 216. Furthermore, detecting the signature pattern of 2D/3D morphology architecture and/or synthetic cognition datascapes by means of signature pattern detection 218, wherein the signature pattern detection helps in visualizing 220 the observable datascapes or observable phenomenon of the 2D/3D morphology architecture and/or synthetic cognition datascapes, wherein the observable phenomenon retains the dynamics/signature of known contextual relationship and provides insights into the working of the whole system without the need to have understanding of its smaller units of function. Moreover, the holistic/aggregate representation of complex multifaceted events when made into the observable format provide new insights into potential outcomes and also behavior of smaller parts. Further, mining and iterating the observable phenomenon/holistic form by conventional and also artificial intelligence and machine learning approaches 222 are followed without deconstruction into smaller parts but as a whole, observable phenomenon.

According to one embodiment herein, FIG. 2A-2C illustrates the stepwise manner details on the formation of D-CELL and D-TISSUE based on geometric organization of D-AXONS. FIG. 2A illustrates the formation of D-AXON 230, wherein the D-AXONS includes data from individual elements converted into a line, where the line width and colour of the data are mapped to disparate attributes of the data. FIG. 2B illustrates the formation of D-CELL 240, wherein the D-CELL is formed when the D-AXONS 230 are arranged in a grid-like pattern in a nodal architecture. A D-CELL 240 is a holistic data matrix of the system at a single timestamp. Furthermore, the FIG. 2C illustrates the formation of D-TISSUE 250, wherein the D-TISSUE is formed as D-CELL 240 timestamps are stacked together to form a large data matrix that captures the whole dynamic or a chronological event of a complex adaptive system.

According one embodiment herein, the FIG. 2D-2G depict a geometric organization of D-AXONs of the present system in a closed or a series configuration. FIG. 2D illustrates a D-CELL 240 formed by geometric organization of the D-AXONs 230 for nodes, node A+C, node A+D, node B+D and node B+C. The D-CELL 240 includes four D-CELL walls 241, 242, 243, and 244, and D-CELL PLASMA (FLUID UNIT) 245 associated with a dynamic internal space flux and responsive to “aggregate” activity of A+B+C+D. FIG. 2E illustrates a prime centroid of the D-CELL 240. The prime centroid 246 is the centre of the D-CELL 240 as per an outer wall. FIG. 2F illustrates a point aggregate 247 of the D-CELL 240, that includes a centroid vector. FIG. 2G illustrates a centre 248 of the inner space, which is an angular representation of aggregate shift of an inner D-PLASMA volume.

According to one embodiment herein, the FIG. 2H illustrates a D-CELL comprising centroid vector representing the relational magnitude and direction. FIG. 2H illustrates a phenomenon 249, wherein each cell has a centroid vector representing the relational magnitude and direction of four variables of the phenomenon 249. Principally, a dynamic complex multi-dimensional system is represented as centroid vector grids among the continuum datascape. The large heterogenous continuum datascape represents multiple contiguous phenomena, wherein some phenomenon is escribed, and some phenomena are un-escribed. The escribed phenomenon is the phenomenon, which is already known, whereas the un-escribed phenomenon is which is not known or disclosed.

According to one embodiment herein, the FIG. 2I illustrates an addition of new grid line/data along the horizontal or vertical direction, corresponding to an exemplary D-CELL. Mathematically, each vector comprises of horizontal and vertical component. The horizontal and vertical components of each D-CELL provide additional value or additional relational information other than the values of the data that forms the D-CELL. Hence, with the addition of one new grid line/data along the horizontal 250 or vertical direction 251, a new cell with relational information along with the existing data is obtained. Furthermore, with the addition of one new grid line/data or variable along the horizontal direction 250 more cells along the vertical direction 251 and vice versa is obtained.

According to one embodiment herein, the FIG. 2J illustrates a graphical representation of horizontal and vertical components of the centroid along with the gridline variables. FIG. 2J illustrates the horizontal and vertical components of the centroids along with the gridline variables. When the gridline variables are plotted as a graph, gives very minimal changes from the previous values as there are three already existing variables. Hence, over the period of time along the horizontal or vertical direction the sensitivity of two common data points becomes prominent.

According to one embodiment herein, FIG. 2K-2L illustrates the juxtaposing of the data in the grid in various combinations. Juxtaposing the position of data variable in the grid provides more insight into un-escribed functions of the phenomenon. FIG. 2K illustrates the data components such as 1, 2, 3, 4, 5, A, B, C, D, E before juxtaposing. FIG. 2L illustrates the data components after juxtaposing. For instance, a phenomenon with data components 1, 2, 3, 4, 5, A, B, C, D, E subsequently after juxtaposing or arranging the data in the grid in various combinations, the resultant grid forms a plurality of patterns, relationships, and smaller phenomenon inside as larger phenomenon. Furthermore, the juxtaposing establishes a new relationship between the escribed and un-escribed data. FIG. 2M illustrates the escribed and un-escribed data relationship. The outcome of juxtaposing function with escribed and un-escribed data yields three types of relationship such as escribed relationship, relationship of potentiality and un-escribed relationship. The escribed relationship is a relationship which is previously known. For instance, in the FIG. 2M the relationship between data components A, B, C and 1, 2, 3 is escribed relationship. The relationship of potentiality is the relationship which yields more information and a new phenomenon. For instance, in the FIG. 2M the relationship between ABC/123 and α, β, γ, ϑ. Besides, the un-escribed relationship is a relationship which is not known. For instance, in the FIG. 2M the relationship between α, β, γ, ϑ, variables depicts the un-escribed relationship.

According to one embodiment herein, FIG. 2N illustrates a D-TISSUE unit 260, corresponding to an exemplary D-AXON 230. FIG. 2N illustrates a D-TISSUE 260 unit. The length of D-AXON 230 retains the same data/speed of transmission of organization of D-CELLS 240 in “Series” allows multiple data sets to be juxtaposed to reveal direct or indirect or absence of correspondence. FIG. 2O illustrates a time chronology square 261 of the D-TISSUE unit 260. The time chronology square includes expansion 262, temperature 263 viscosity 264, and a non-specific sensor input 265.

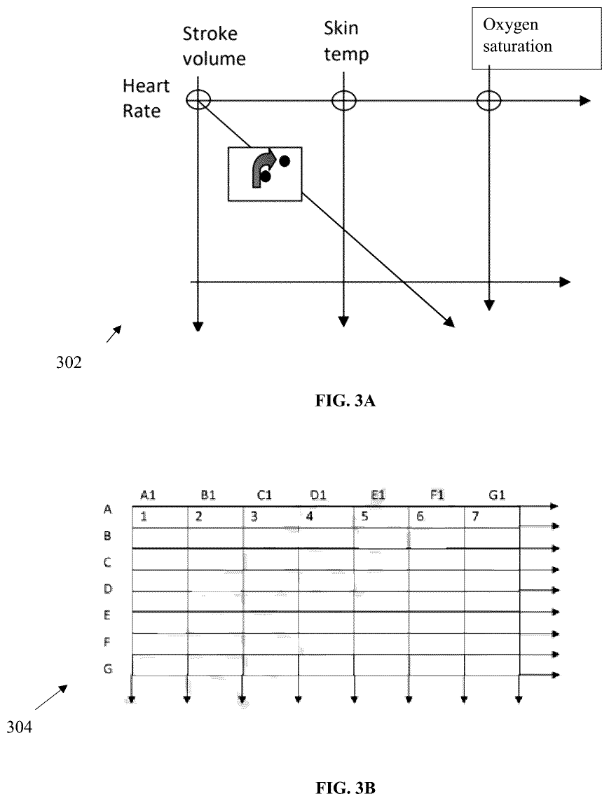

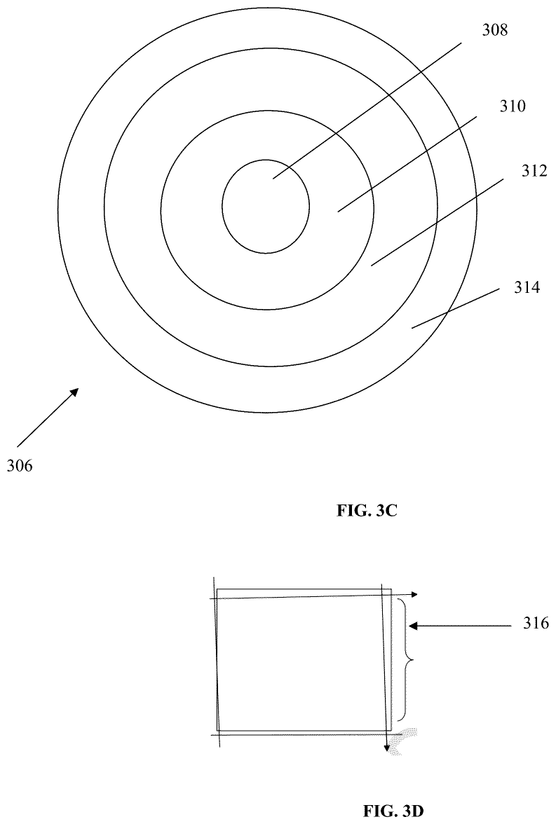

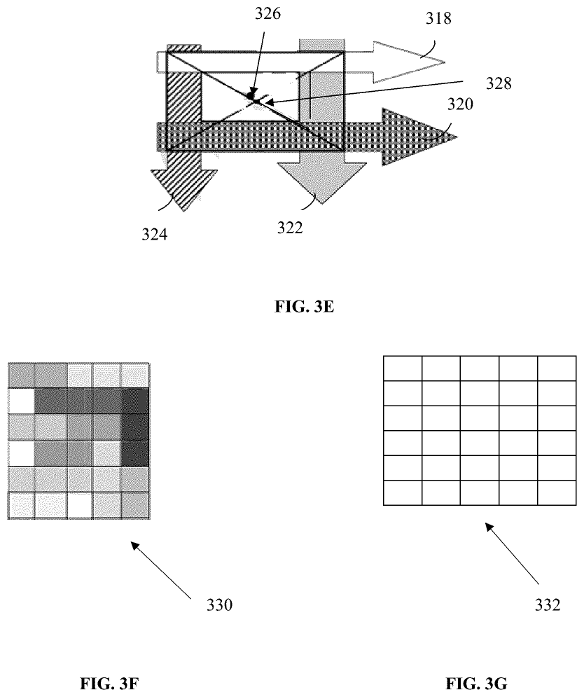

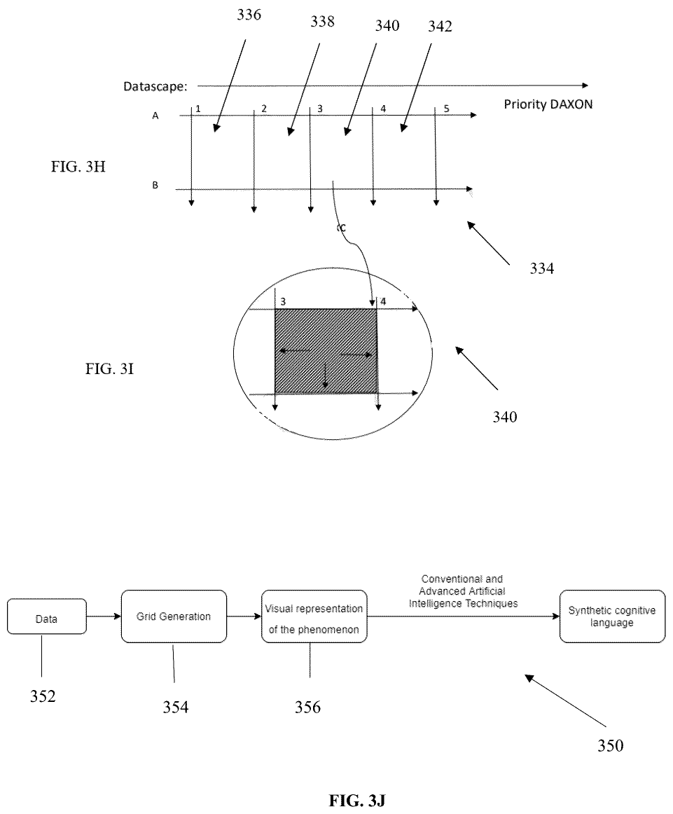

According to one embodiment herein, the FIG. 3A illustrates a derivative function versus saturation plot indicating an aggregate impact of blood pressure, skin temperature and oxygen saturation. FIG. 3A illustrates a derivative function (Y-AXIS) versus saturation (X-AXIS) plot 302 indicating aggregate impact of blood pressure, skin temperature and oxygen saturation in an exemplary scenario. The nodal characteristic expresses blood pressure flux given by equation (1) Stroke volume*heart rate=blood pressure.

According to one embodiment herein, the FIG. 3B illustrates a clustered data and sequentially aligned data as cluster of known maximal conformity. FIG. 3B illustrates pre-processing data 304. A pre-processing data is used to create parity. More particularly, FIG. 3B illustrates a clustered data and sequentially aligned data as cluster of known maximal conformity, such as for example fluids relational categories, demonstrating in the studies systems a known relationship moving towards the least coincident activity.

According to one embodiment herein, FIG. 3C illustrates a nodal organization hierarchy. FIG. 3C illustrates a nodal organization hierarchy 306 in accordance with an embodiment. The nodal organization hierarchy 306 includes an inner most zone of direct activity 308, a zone of potentiality 310, a zone of probability 312, and a zone of enquiry 314 (outermost zone). The system of the present technology enables the cells to be migrated as per activity and understanding towards the centre.

According to one embodiment herein, FIG. 3D illustrates D-CELL dimension determinants. FIG. 3D exemplarily illustrates D-CELL dimension determinants. As depicted in FIG. 3D, the D-CELL 240 includes a D-CELL wall 316, that includes a minimum length required to accommodate a data flux in a territory as assigned.

According to one embodiment herein, FIG. 3E illustrates a maximal extent of data flux determination. FIG. 3E illustrates a maximal extent of data flux determination, in accordance with an exemplary scenario. More particularly, FIG. 3E illustrates intersection of four D-AXONs (data D-AXONs) A, B, C and D 318-324. FIG. 3E also illustrates a D-CELL plasma 326 and a D-CELL wall centroid 328. An angle and direction of drift from D-CELL plasms 326 and D-CELL wall centroid 328 indicates a net activity of the four D-AXONs.

According to one embodiment herein, FIG. 3F-3I illustrates a D-CELL Plasma centroid shift surrogate for net activity. FIG. 3F-3I illustrates a D-CELL plasma centroid shift surrogate for net activity. In an embodiment, the datascape can include contraction or expansion. A thermogram is determined based on a determination of a relative rate of concentration and a D-CELL stress. FIG. 3F illustrates a thermogram 330 of D-CELL plasma and FIG. 3G illustrates a cell accommodation 332 of D-CELL. The D-CELL wall dimensions are assigned, and D-CELL wall dimensions are computed and correspondence, lack of correspondence, a flux accommodation for territorial organization are categorized. Some data flux changes are so large that the transition demands D-CELL enlargement (DCELL hypertrophy) when the correspondence of all four datasets (D-AXONs A-D 318-324) within the cluster remains unaltered or a little altered. The D-CELL accommodation 332 includes accommodation of a phase transition and accommodation of an emergent behaviour. FIG. 3H illustrates an exemplary datascape 334. The datascape 334 includes various D-CELLs 336-342 and includes aggregates of known (escribed) and unknown (un-escribed) activity into map as datascape cells. FIG. 3I illustrates an exemplary D-CELL hyperplasia of the D-CELL 340. The D-CELL hyperplasia includes addition of new D-AXONs or accommodation of increasing activity by one or more D-CELLs by incorporating D-CELL of hyperactivity and a territorial expansion.

According to one embodiment herein, FIG. 3J illustrates the process steps involved in development of synthetic cognitive language to represent static/dynamic/continuum data, observable phenomena integrable into computational system. The synthetic cognitive language is a representation of the whole phenomenon including all the static, dynamic, and continuum data. The steps involved in development of synthetic cognitive language 350 includes collating data 352, using the gathered data to generate the grid 354, visual representation of observable phenomenon 356 by means of analysing the generated grid 354. Finally using conventional and advanced artificial intelligence techniques to develop the synthetic cognitive language 350. The synthetic cognitive language can be assigned not only on the basis of known or ascribed relationships or design but also un-ascribed. Furthermore, the synthetic cognition language can also be used to capture data and represent the data into computational systems known in the art.

The various embodiments of the system of the present technology transforms heterogeneous data into observable phenomenon that can be taxonomically and in other ways organized, in addition to conventional factors involved in data analytics and looking for connections the entire phenomenon is rendered demonstrable.

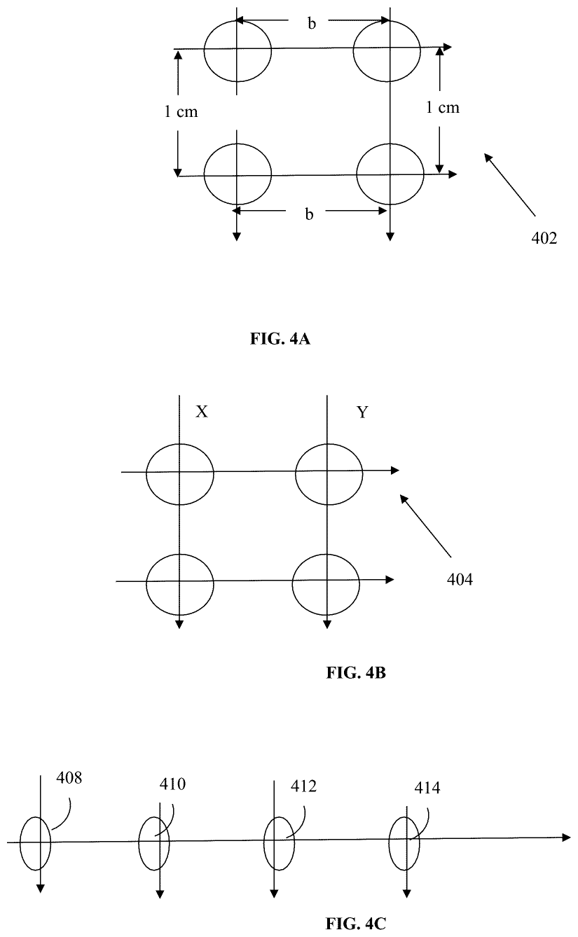

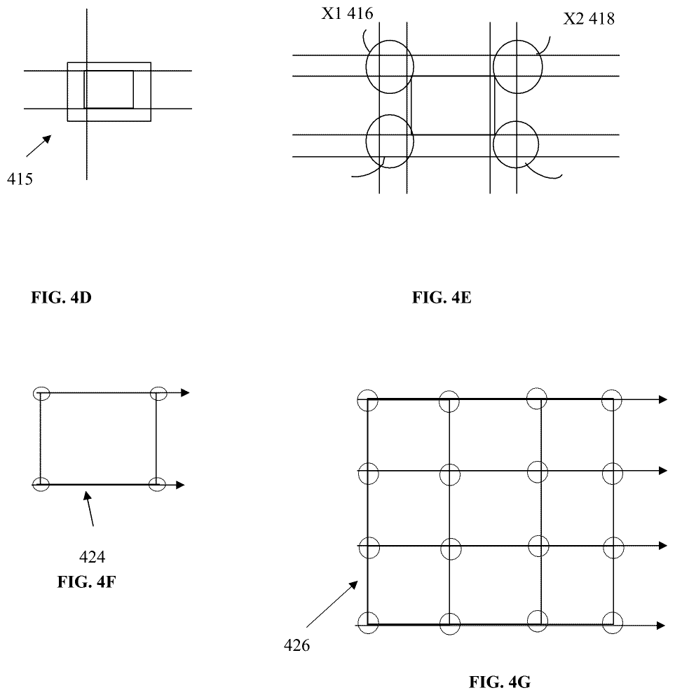

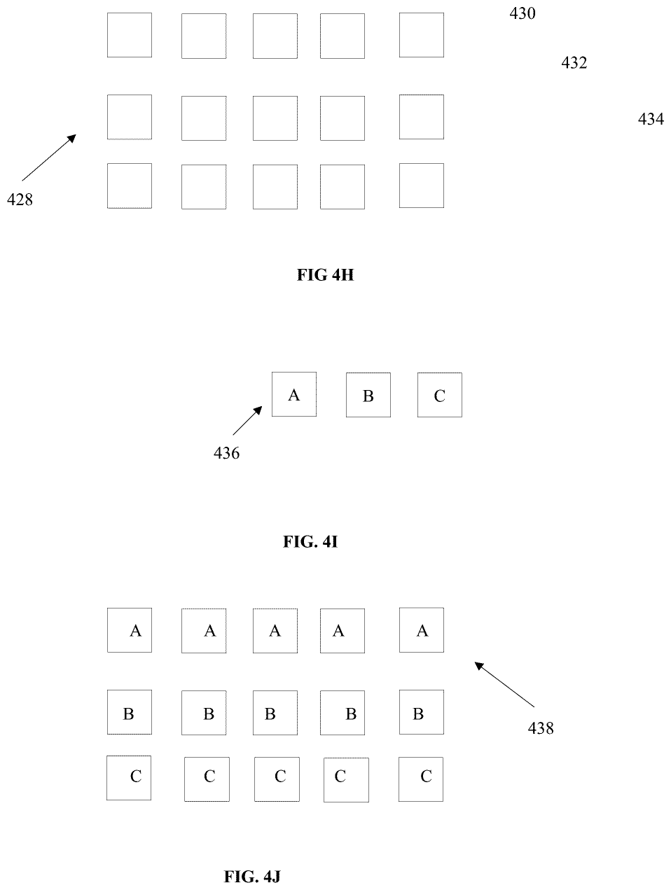

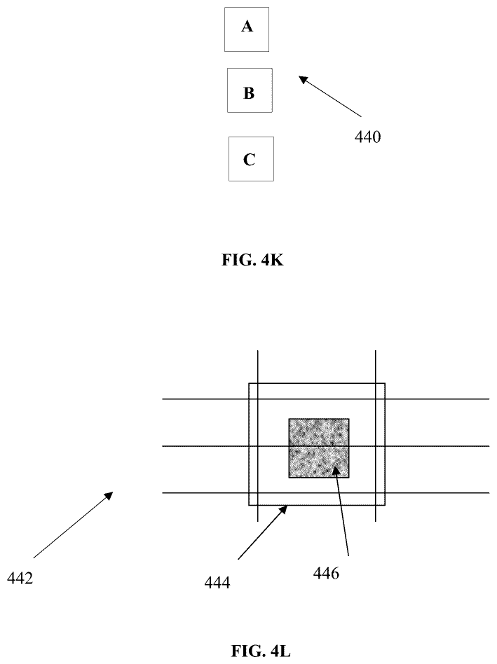

According to one embodiment herein, FIG. 4A illustrates an exemplary taxonomic organization of an exemplary cell unit in accordance with an embodiment. The cell unit 402 of a cell size of one square centimetre, accommodates flux in all the four D-AXON data sets. According to concordance and according to function or proportionate flux the ascribed value, conformity sets boundary characteristics. FIG. 4B illustrates an exemplary D-CELL 404 where each node represents measurable known functional relationship. In an exemplary scenario, a complimentary data (A) 406 is given by equation (2):

Complimentary data (A)=heart rate+stroke volume

X and Y bisecting the complimentary data (A) could be peripheral pulse values which would be concordant (similar) but not the same.

According to one embodiment herein, FIG. 4C illustrates a series of nodes 408-414. In the series of nodes 408-414, each node is relational to blood pressure. The blood pressure is given as a combination of heart rare and stroke values. All the nodes 408-414 together form one data set and are interchangeable. One or more basic (first level) discriminatory units connect the four data point. In an embodiment, a minimum or a maximum inner and outer limit is selected. FIG. 4D illustrates convergent aggregate impact of four datapoints. FIG. 4E illustrates an aggregate impact of four nodes and eight datapoints. The four nodes X1-X4 416-422, each gives contribution of one new datapoint. The D-CELL optimization is performed by the system of the present technology, based on datascape of organization in a variety of modes, providing alternative datascapes for alternative analysis in a different manner. The D-CELL optimization is performed based on 1) known functional significance (function to unknown), 2) optimal synchronicity on the basis of system, 3) predictive modelling, random dynamic plans (situational), event representation, and the like. FIG. 4F illustrates an example D-TISSUE 424 in accordance with an embodiment. The D-TISSUE 424 includes a collection and organization of data cohorts of variable sizes. The D-TSSUE 424 depicted in FIG. 4F particularly includes four units of cohorts. FIG. 4G illustrates an example D-TISSUE 426 with 78 units of cohorts (not completely shown) 4G illustrates 8 data points 8, and 16 nodes.

According to one embodiment herein, FIG. 4H illustrates a linear organization of D-TISSUE 428, in accordance with an embodiment. The linear organization of D-TISSUE 428 can be in the form of a single layer 430, 2 layers 432, or 3 layers 434. Each cohort in the linear organization of D-TISSUE 428 includes data cohort of similar data, including for example pressure, temperature and the like and each cohort can be of 4 units, 8 units and the like. For example, 8 different data making up each unit of big data enquiry makes up and is represented by one D-CELL. Similar D-CELLS representing multiple subjects create a large canvas of interconnected but independently responding D-TIS SUE. Each D-CELL in the linear organization of the D-TISSUE 428 retains its independent properties. The behaviour of the D-TISSUE 428 is collated to identify similarities complementarity and divergence and the like, to determine “polarity” and “placement” of cells in the organization of the D-TISSUE architecture either as a means of adaptive system creation or causality determined by the D-TISSUE organization. Consider ‘n’ to be the number of datapoints and ‘d’ be the total number of D-CELLS, then d is given by equation (3):

d=((n−2)/2)2 (3)

for even number of datapoints having values >=4.

For odd number of datapoints having values >=5, d is given by equation (4): d

=t*(t−1) (4)

Where T is a temporary variable given by equation (5):

t=(n−1)/2 (5)

For example, if the number of datapoints is 8, then d=((8−2)/2)2=9.

If the number of datapoints is 7, then first we need to calculate t which is (7-1)/2=3, then d=3*(3-1)=6.

According to one embodiment herein, adaptive is the natural organization based on taxonomic or other forms of categorization into an evolving hierarchy of D-CELLs while causality-based organization is for example, a planned outcome that is predetermined and achieved as in the case of strategic management of healthcare from illness D-signature to a wellness D-signature either in relation to human health or that of a complex engine system, automation, or society. In an embodiment, the categories of D-CELLs which evolve towards a more stable observable performance state or an ascribed causality-based D-CELL characteristics indicates a differentiation into a specific category similar to a skin cell can change into a specialized pigment bearing cell which as its numbers increase becomes a phenomenon as a visible coloured mole. Similarly, even quantitative, and qualitative data.

According to one embodiment herein, the cohorts are categorized as per performance into various categories 436 such as A, B, and C categories as depicted in FIG. 4I. FIG. 4J illustrates an example of D-CELL stratification 438 resulting in an adaptive tissue system. The D-CELL moves towards a “differentiation” in terms of selective use of application of specific data sources thereby resulting gradually into the architecture of a D-CELL cohort into an optimal D-TISSUE configuration. For example, consider a D-CELL from soldiers' performance, a D-CELL A is associated with a category including foot soldiers and a D-CELL B is associated with a category infantry soldiers. Both D-CELL A and D-CELL B are optimized for data selection. D-CELL cohorts have a simple linear organization, collating a selection, D-CELL differentiation categories. D-CELL moves away to a new D-COHORT category when a particular performance matrix in D-CELL cohorts demonstrates consistent/stable. D-CELL specific performance retention of D-CELL category as a type. Since data cohort attributes are similar but performance characteristics differ. It becomes possible based on data attributes organize D-CELLs into stratification as stratified D-CELLS forming a D-TISSUE.

According to one embodiment herein, FIG. 4K illustrates an exemplary organization 440 of categories of cohorts according to stage of change, in accordance with an embodiment. The state of change can be for example change of amplitude. As depicted in FIG. 4K, the cohort can be categorized in progression to next level of organization into architecture of cohort tissue, where the categories can include a category C corresponding to most frequent state of change, a category B corresponding to intermediate level of frequent state of change, and category A corresponding to a stable state of change.

According to one embodiment herein, a data cluster includes collection of one or more small number of D-CELLS closely connected to measurable/known phenomenon where the activity of the cluster visually represents largely predictable events. FIG. 4L illustrates an exemplary data cluster 442, including an outer envelope 444 and an internal volume flux 446. By simple adjustment in orientation of the data sets enables estimation of relative values in multiple functional settings. Since performance is the outcome of aggregate activity of multiple datasets working optimally, the data cluster dynamics enables shifts in relationship of datapoints and its impact on the overall (aggregate/holistic) performance. The status of the outer envelope 444 and the internal volume flux 446 (representing the internal space changes of each of the D-CELLs in a given organization) is an indicator of the aggregate performance.

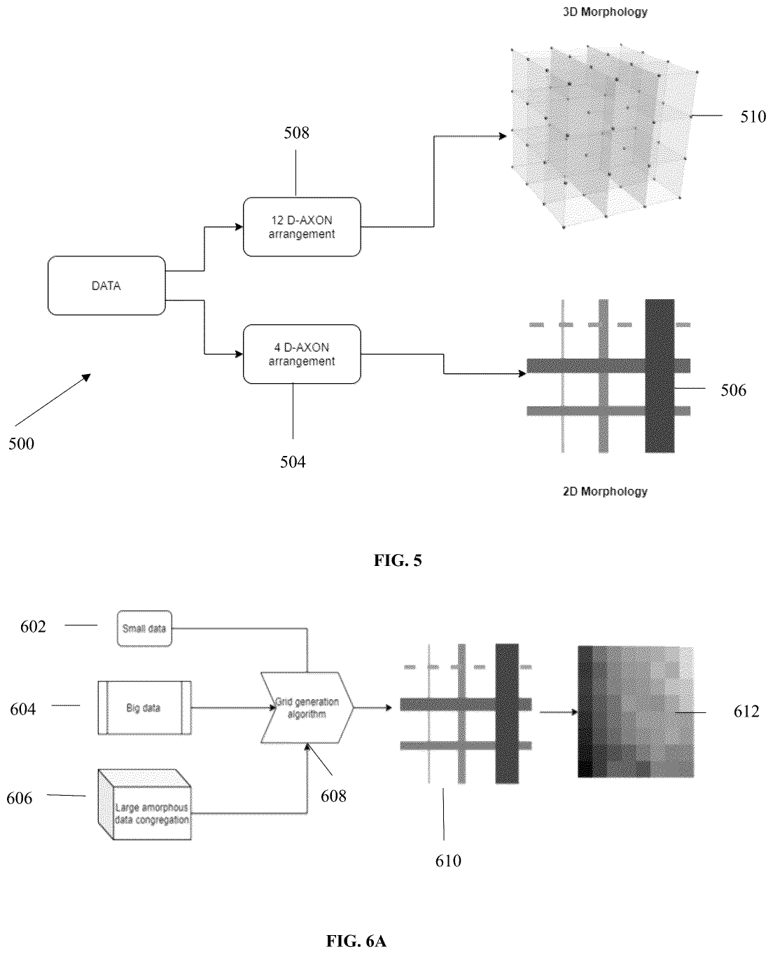

According to one embodiment herein, FIG. 5 illustrates the process steps involved in method to build data aggregates into interconnected passive or dynamic constructs as building units for the creation of 2D/3D morphology architecture and/or synthetic cognition datascapes enabling delineation or territorial distinctions. FIG. 5 illustrates method to build 2D and 3D datascapes 500 from data 502. Initially data 502 is converted into D-AXONs (504, 508) by means of grid conversion algorithm, wherein D-AXON consists of data from individual elements converted into a line, where the line width and colour of the data are mapped to disparate attributes of the data. The D-AXONs (504, 508) thus obtained creates both 2D 506 and 3D 510 morphologies. The Grid in 2D 506 gives an interconnected dynamic relationship between four Axons 504 in one cell. Similarly, Grid in 3D 510 gives an interconnected dynamic relationship between twelve Axons 508 in one cell.

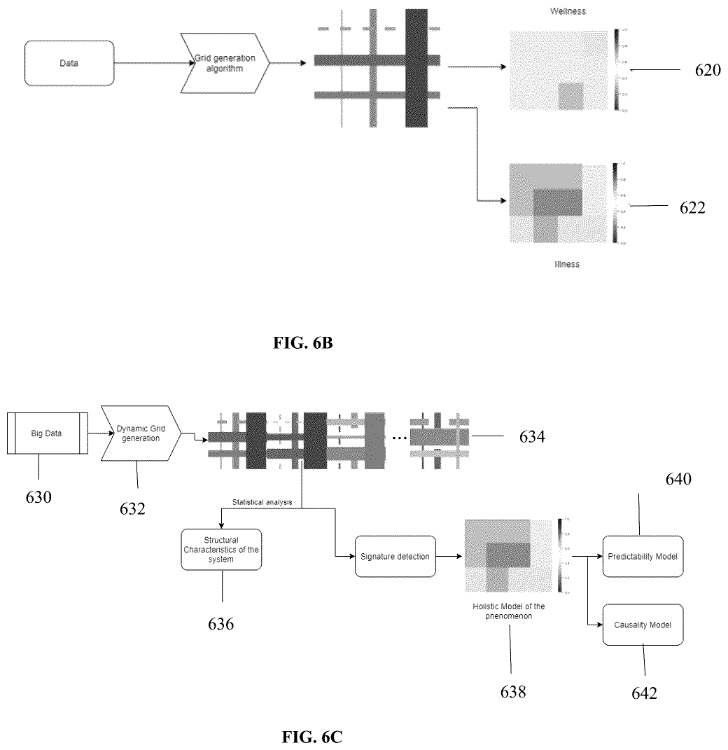

According to one embodiment herein, FIG. 6A illustrates the method to transform different types of data into “observable phenomenon” that retains dynamic signatures. FIG. 6A illustrates a method to transform small data cohorts 602, big data 604 and large amorphous data congregations 606 into an observable phenomenon 612 by means of grid generation algorithm 608, wherein the observable phenomenon 612 retains dynamic/signature of known contextual relationships and further the observable phenomenon 612 is integrated within a larger contiguous data scape or as part of a heterogeneous composite functional system, as “observable” but unidentified or un-ascribed discoveries. The grid generation algorithm 608 creates 2D grid 610 with contextual relationship between larger contiguous data. The contextual relationships of the elements of the system are integrated within the large contiguous data scape, or as part of a heterogeneous composite functional system. Hence, the observable phenomenon 612 but unidentified or un-ascribed discoveries can be detected by signature patterns and progression pathways even before the endpoint is observed.

According to one embodiment herein, FIG. 6B illustrates the method to capture broad holistic outcomes in clearly identifiable broad categories in biological or non-biological system. FIG. 6B illustrates the method to capture broad categories outcome such as wellness and illness or harmony and disharmony in biological or non-biological system with access to a reverse approach to iteration and/or manipulation without necessarily having to divide into small constituent components for detailed analysis. Broad and clearly identifiable categories of change like wellness 620 and illness 622, harmony and disharmony, best, better, good, bad, worse, and worst are manifested from observing data as a holistic interconnection of a complex dynamic adaptive system. The changes in the data when represented individually are predictable only when the data crosses its threshold of detection. Hence, when the data is observed as a whole, the changes due to any structural modifications are manifested first and the functional changes follow the structural manifestations. Thus, enabling the new possibilities of surveillance, predictability, and causal model.

According to one embodiment herein, FIG. 6C illustrates the method for testing “causality” and “predictability”. FIG. 6C illustrates the method of mapping big data 630 as dynamic interconnected large datascapes 634 created by means of dynamic grid generation 632. The dynamic interconnected large datascapes 634 demonstrates real-time values and structural characteristics 636 captured by statistical and/or other modes of analysis and functions as a holistic model 638 for displaying predictability like wellness 640 and testing models of causality like illness 642.

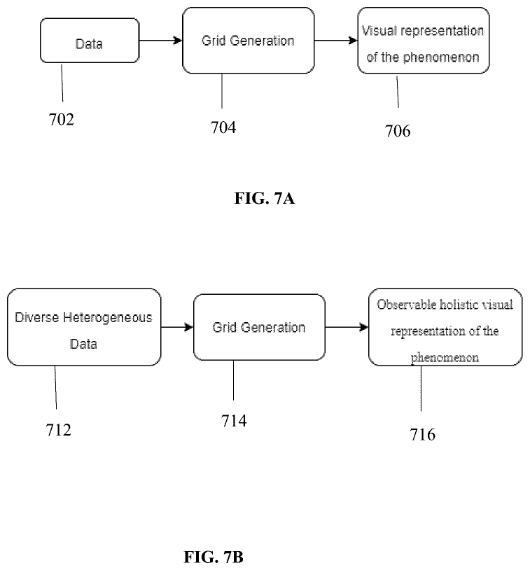

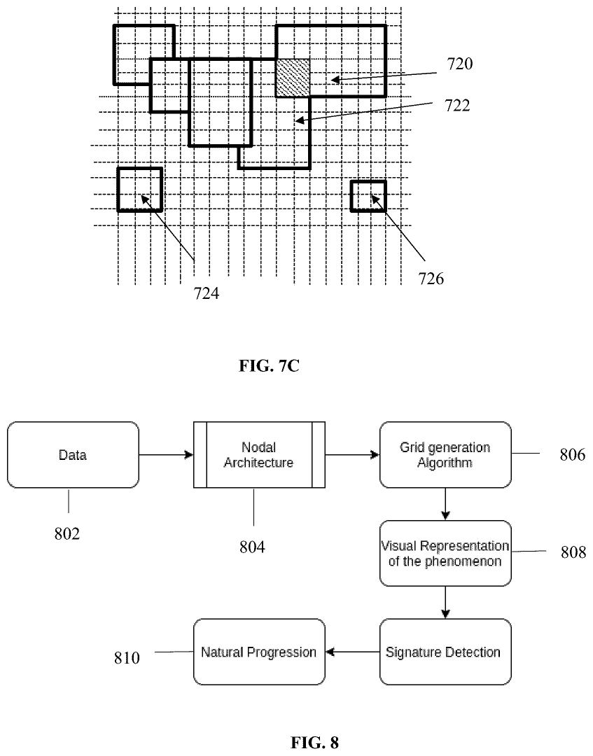

According to one embodiment herein, FIG. 7A illustrates the method to transform multiple data aggregations into an “observable phenomenon”, which provides insight into working of entire system. FIG. 7A illustrates the visual representation of the phenomenon 706 enables the manifestation of broad categories of changes. When the data 702 is observed as a visual representation of the phenomenon 706, the changes due to any structural modifications are manifested first and the functional changes follow the structural manifestations. Thus, eliminating the need to understand the small functional units individually in order to understand the entire system. The data 702 is observed as visual representation 706 by means of grid generation algorithm 704.

According to one embodiment herein, FIG. 7B illustrates the method to transform diverse heterogenous data into observable holistic representation. FIG. 7B illustrates diverse heterogeneous data 712 or multiple data aggregations is transformed into an observable composite/holistic form 716 by means of grid generation algorithm 714, wherein the observable composite/holistic form 716 can be mined or analysed by a number of methods such as topology, artificial visualization, and optical/pixel-based analytics without deconstruction into smaller parts but as a whole, the multiple data aggregations. Therefore, when the data 712 is converted into an observable visual medium 714, various topographical analysis can be performed to understand the territorial changes, artificial visualization, and pixel analytics can be performed to capture the dynamic change in the signatures due to structural or functional changes in the complex adaptive system.

According to one embodiment herein, FIG. 7C illustrates a topological organization of the datascape. The datascape can be used in various applications, such as industry performance, socioeconomics, healthcare politics, psychology, robotics, and the like. The datascape represents a large collection of similar or same activity cells, e.g., each cell or cluster indicating a person's accessible data and topographic organization relating to people in an industry, geographic location, and the like. The topological organization and analysis provide insights. The topological organization of FIG. 7C illustrates independent envelopes 720 and 722, overlapping envelopes 724 and 726, isolating envelopes and the like, forming the clusters/cells within the datascape.

The topological organization includes dynamic representation of performance dynamics of complex industry, processes, evolutionary molecular and other phenomenon into a visual mapping encompassing both known as well as not yet known data values as documentation of temporal status or analysable change. This enables documentation (e.g., of diverse psycho-social, economic, political, and the like) states at a given time or a record of flow of events across time or progress towards optimization or decline as it relates to its parts.

In a political survey representing a large population cohort, a dynamic datascape helps to directly link confessed political choices to related accessible factors for demographics to relative spending, health, psychological status, and the like. Critically analysable cell, cluster, datascapes can be translated or applied to any dynamic phenomenon to obtain a signature based on aggregate performance in a complex system of any other representational data, such as a new paradigm of “M biography.” M-biography represents a new paradigm of “Wellness” (positive health balance) or “Illness” (negative health balance). An M-biograph is normal/stable or wellness and is perturbed, unstable or abnormal for illness. The M-biograph clusters are associated with various data categories, such as cell phone based (e.g., cognitive, psychological, motor, performance routine, sleep patterns, and the like), wearable bio-sensor based data (e.g., heart rate, oxygen, breathing, perfusion, temperature, non-specific sensors along the body, and the like), and external data (e.g., movements, facial features, predictability of daily routine, sensors in home and in community, and the like). Amplification and objectivization of normal subjective feelings of wellness and illness so that analysis and discrimination into potential domains such as for example, metabolic etc., becomes manifest independent of specific diagnosis of disease.

According to one embodiment herein, FIG. 8 illustrates a method for organizing the observable data into an architecture that results in natural progression. FIG. 8 illustrates organizing the data 802 into a nodal architecture 804 that results in the natural progression 810 towards organization both as a “new phenomenon” in addition to those deducible by knowledge of parts for example, blood pressure, heart rate, and the like. The natural progression of the phenomenon 810 is achieved by means of grid generation algorithm 806, wherein the grid generation algorithm 806 generates visual representation of the phenomenon 808, which in turn produces natural progression 810. The reductionist approach on the system by deducing the system into known parts will not give a complete understanding of the system or the phenomenon. Different pathways of progression lead to the same endpoint. Therefore, by deducing the knowledge to the parts of the system can help understand the endpoint but not the cause whereas the integrated approach helps to understand the natural progression of the phenomenon.

According to one embodiment herein, the system and method of the present technology is also applicable in robotic sensorium/robotic humanization. Robotic sensorium (cybernetics) is an envelope of perception that encompasses the body to enable a dimension of knowledge of one's immediate surroundings (as compared to eyes, ears, etc.) and nature of contact (physical). While touch, pressure, perception, and temperature constitute human biological characteristics, electrical (electrodermal) electromagnetic, etc., in robotics can provide viable surrogates. These exhibit territorial and specialist attributes enabling identification of the origin of the sensation characteristics and significance. The datascape provides an excellent mechanism of representation, analysis, storage, discrimination, and gating.

In various embodiment, in large datascapes where each D-AXON line can be linked to a specific source-based data set and where every D-CELL share datapoints with its neighbour, the whole datascape constitutes a Data-syncytium. In an embodiment, multiple similar cohorts that are variable in terms of performance constitute a D-mosaic. For example, each D-CELL or D-CELL cluster represents data from a single individual and the D-Mosaic constitute a population. The present technology enables using a biologically inspired engineering construct as the basis for development of a complex system as an organism having a hierarchy start from a single D-CELL to simple aggregations as a D-Tissue to a D-Organ construct which either represents the actual systems organization of a source or a derived organization ascribed from categorizing D-CELLs based on performance within the phenomenon.

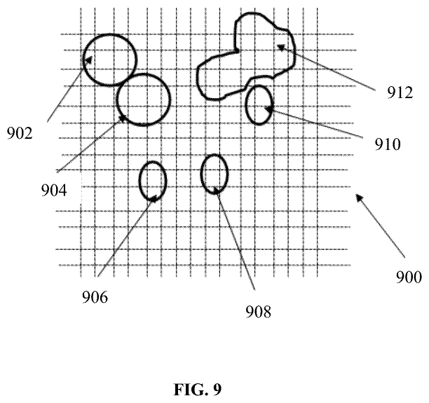

According to one embodiment herein, FIG. 9 illustrates an exemplary datascape of cyber sensorium. The datascape 900 of FIG. 9 includes variability in space allocation and relation of overlapping and proximate territorial envelopes 902-912, that provides information about origin of data from the sensorium.

Skin in biological design is an extension of the brain that provides instant sensory input from every external part of the body and enables it to be variably expressed according to need. Entry system for categorization and prioritization receive touch, pressure, proprioception, temperature, and pain (fast or slow) as inputs and selects a destination. The destination includes, for example, grids (concept of superimposed grids), memory (neural reinforced), affective (value-based superimposition), response (immediate, intentional, decision and the like), and cognition as sensation.

The Various embodiments herein provided can be used in a) multiple categories of economic information that make up aggregate values such as the gross domestic product, b) clusters of disparate activity that characterize a sophisticated manufacturing process, c) cumulative impact of several factors that collectively signal the state of health as wellness or non-wellness ascribed to each individual and linked to changes related to lifestyle, environment or ageing that predate the diseases that ultimately result from it, and d) optimal state of mechanical harmony collectively representing optimal functioning of a multisystem machinery, and the like.

Although the embodiments herein are described with various specific embodiments, it will be obvious for a person skilled in the art to practice the embodiments herein with modifications.

The synthetic cognition (SC) intuitive system and method for predicting, analysing, and managing complex adaptive and non-adaptive system in engineered or partially biological application disclosed in the embodiments herein have several exceptional advantages. The system and method that uses a synthetic-cognition language to represent static/dynamic/continuum data, is integrable into the computational systems that facilitates understanding of dynamic complex multi-dimensional systems as territorial clusters among a continuum datascape. Further, the embodiment, also enables translating diverse data into datascapes based on the synthetic cognition language and also provides a mechanism for organization of diverse data for the study of complex phenomena. The embodiment builds data aggregates into interconnected passive or dynamic constructs as building units for creation of multi-dimensional morphology and/or synthetic cognition datascapes enabling delineation of territorial distinctions. Additionally, the embodiment facilitates translation of datascapes into subjective surrogates of biological cognitive phenomenon, as objective substitutes for biological awareness. Moreover, the embodiment enables synthetic cognition as tool for diagnosability and predictability in engineered/nonbiological or partially biological systems. The embodiment also provides an alternative computational system for data analytics. The embodiment enables D-TISSUE organization into observable phenomena of complex systems that can be studied independent of specific smaller units. The embodiment provides a holistic/aggregate representation of complex multifaceted events which when made into an “observable phenomenon” provide new insights into potential outcomes and also behaviour of smaller parts such as for example, study of a phenomenon such as a tornado provides valuable information about known contributing components such as wind speed, but also new unanticipated data such as geography and the like.

The embodiments herein also provide systems for organizing the observable data into an architecture that results in natural progression towards organization both as a “new phenomenon” in addition to those deducible by knowledge of parts for example, blood pressure, heart rate and the like. Furthermore, the embodiment has diverse applications in economics, big data, health, and wellness/illness. The embodiment behaves remarkably similar to the human cognition where we do not need to track our heart, or liver but only know and need to recognize if we are well or ill and then work out in which area potentially is the seat of infirmity.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications. However, all such modifications are deemed to be within the scope of the claims.

What is claimed is:

1. A system for predicting, analyzing, and managing complex adaptive and non-adaptive system in engineered/non-biological, or partially biological applications using synthetic cognition or intuitive machine learning approach, comprising;
a data collection module configured to capture and collate a plurality of data and to route the captured plurality of data, and wherein the plurality of data is an asynchronous data and comprises small data, big data, and large amorphous data congregation;
a data processing module configured to receive the plurality of captured data from the data collection module;
a data cleaning module provided in the data processing module and configured to clean up the plurality of asynchronous data and to convert the asynchronous data into synchronous data;
a data encoding module provided in the data processing module and configured to encode the synchronous data;
a normalization module provided in the data processing module and configured to analyses the dynamic statistics of the synchronous data and to normalize the synchronous data qualitatively and quantitatively;
a grid generation module configured to seamlessly integrate the normalized synchronous data into a dynamic matrix using a synthetic cognitive language so as to capture a datascape, and wherein the datascape comprises a holistic working of complex dynamic-adaptive functions, and wherein the grid generation module is configured to perform categorization of data in nodal architecture by classifying the data into four clusters, and wherein the four clusters comprises a zone of direct active, a zone of potentiality, a one of probability and a zone of enquiry, and wherein the grid generation module is further configured to convert the categorized data in nodal architecture into interconnected passive or dynamic constructs, and wherein the dynamic constructs are building units for the creation of two-dimensional (2D)/three-dimensional (3D) morphology architecture and/or synthetic cognition datascapes for enabling delineation of territorial distinctions by means of grid conversion algorithm;
a data mining module configured to receive the 2D/3D morphology and datascapes from the grid generation module to assess or visualize the grid generated datascapes and 2D/3D morphology, by analyzing or detecting the signature patterns of datascapes through a signature pattern detection algorithm, and wherein the data mining module is further configured to help in mining and iteration of the datascapes using artificial intelligence and machine learning techniques.

2. The system according to claim 1, wherein the synthetic cognitive language enables surrogate process of apprehension of knowledge and understanding to create a dynamic data landscape that reflects a totality of inputs which is mined for specific information, and the synthetic cognitive language is a representation of an entire phenomenon including all the static, dynamic, and continuum data, and wherein the synthetic cognitive language is assigned not only on the basis of known or ascribed relationships or design but also on the basis of un-ascribed, and wherein the synthetic cognition language is employed to capture data and represent the data into computational systems.

3. The system according to claim 1, wherein the zone of direct active of nodal architecture comprises of data having a deterministic or directional relationship between a plurality of elements/parameters, and wherein the plurality of elements or parameters is selected from a group consisting of dynamic viscosity, Temperature, Pressure, Reynold's number, expansion, volume flow rate, cross-sectional area and dynamic friction co-efficient, and wherein the deterministic or directional relationship between the elements is defined by a formula or an equation.

4. The system according to claim 1, wherein the zone of potentiality of nodal architecture comprises data having a surrogate relationship between the plurality of elements, and wherein the plurality of elements does not have a direct equation to relate these elements but have a linear relationship between these elements

5. The system according to claim 1, wherein the zone of probability of nodal architecture comprises of data having a probabilistic cause-effect relationship between the elements.

6. The system according to claim 1, wherein the zone of enquiry of nodal architecture comprises of data having a possible cause-effect relationship between the elements, and wherein the cause-effect relationship is not yet defined by any theorem, and wherein the relationship is unknown and is only manifested when the changes are observed between the elements along with the other categories.

7. The system according to claim 1, wherein the building units for the creation of two-dimensional (2D)/three-dimensional (3D) morphology architecture and/or synthetic cognition datascapes generated via grid generation module includes D-AXONS, D-CELLS and D-TISSUES, and wherein the D-AXONS includes data from individual elements converted into a line, and wherein the line width and colour of the data are mapped to disparate attributes of the data, and wherein the D-CELL is formed when the D-AXONS are arranged in a grid-like pattern in a nodal architecture, and wherein the D-CELL is a holistic data matrix of the system at a single timestamp, and wherein the D-TISSUE is formed as D-CELL timestamps are stacked together to form a large data matrix that captures the whole dynamic or a chronological event of a complex adaptive system, and wherein a plurality of complex adaptive systems stack to form even a larger data matrix of D-ORGAN and D-ORGANISM respectively, and wherein the dynamic relationship between the cells arranged in a 2D or 3D morphology enables to delineate the changes occurring together in which the data is related structurally or functionally.

8. The system according to claim 1, wherein the signature patterns detected by the data mining module include machine learning algorithms and artificial intelligence to identify a result of the analysis of the complex adaptive systems into identifiable categories comprising wellness and illness, harmony and disharmony, best, better, good, bad, worse, and worst, and wherein the datascapes are visualized via data mining module and the changes due to any structural modifications are manifested first and the functional changes follow the structural manifestations, thereby eliminating a need for an understanding through a reductionist approach of studying small functional units individually in order to understand the whole system.

9. The system according to claim 1, wherein the mining and iteration of the datascapes by artificial intelligence and machine learning algorithms includes pattern recognition, image classification, Generative Adversarial Network, Convolutional Neural Network.

10. A computer implemented comprising instructions stored on a non-transitory computer readable storage medium and executed on a hardware processor in a computer system for predicting, analyzing, and managing complex adaptive and non-adaptive system in engineered/non-biological, or partially biological applications by using synthetic cognition or intuitive machine learning techniques through one or more applications/algorithms, the method comprising the steps of:
capturing and collating plurality of data with a data collection module, wherein the plurality of data are asynchronous data and comprises of small data, big data, and large amorphous data congregation;
cleaning up the captured and collated plurality of data with a data cleaning module;
converting the plurality of asynchronous data into synchronous data with the data cleaning module;
encoding the synchronous data with a data encoding module;
analyzing the dynamic statistics of the synchronous data with a data processing module;
normalizing the synchronous data qualitatively and quantitatively with a normalizing module;
categorizing the normalized synchronous data in a nodal architecture with the normalizing module;
integrating the normalized synchronous data into a dynamic matrix using a synthetic cognitive language with a grid generation module seamlessly to capture a datascape, and wherein the datascape comprises a holistic working of complex dynamic-adaptive functions;
converting the categorized synchronous data into interconnected passive or dynamic constructs with the grid generation module and wherein the interconnected passive or dynamic constructs are building units/blocks for creating of two-dimensional (2D)/three-dimensional (3D) morphology architecture and/or synthetic cognition datascapes thereby enabling delineation of territorial distinctions through a grid conversion algorithm;
categorizing data in nodal architecture with the grid generation module by classifying the data into four clusters, and wherein the four clusters comprises a zone of direct active, a zone of potentiality, a one of probability and a zone of enquiry, and wherein the grid generation module is further configured to convert the categorized data in nodal architecture into interconnected passive or dynamic constructs, and wherein the dynamic constructs are building units for the creation of two-dimensional (2D)/three-dimensional (3D) morphology architecture and/or synthetic cognition datascapes for enabling delineation of territorial distinctions by means of grid conversion algorithm;
detecting the signature pattern of 2D/3D morphology architecture and/or synthetic cognition datascapes with a datamining module using a signature pattern detection algorithm, wherein the signature pattern detection helps in visualizing the observable datascapes or observable phenomenon of the 2D/3D morphology architecture and/or synthetic cognition datascapes, wherein the observable phenomenon retains the dynamics/signature of known contextual relationship and provides insights into the working of the whole system without a need to have understanding of its smaller units of function, and wherein the holistic/aggregate representation of complex multifaceted events when made into the observable format provide insights into potential outcomes and also behaviour of smaller parts;
mining and iterating the observable phenomenon/holistic form by artificial intelligence and machine learning techniques without deconstruction into smaller parts but as a whole, observable phenomenon.

11. The method according to claim 10, wherein the synthetic cognitive language enables surrogate process of apprehension of knowledge and understanding to create a dynamic data landscape that reflects a totality of inputs which is mined for specific information, and the synthetic cognitive language is a representation of an entire phenomenon including all the static, dynamic, and continuum data, and wherein the synthetic cognitive language is assigned not only on the basis of known or ascribed relationships or design but also on the basis of un-ascribed, and wherein the synthetic cognition language is employed to capture data and represent the data into computational systems.

12. The method according to claim 10, wherein the zone of direct active of nodal architecture comprises of data having a deterministic or directional relationship between a plurality of elements/parameters, and wherein the plurality of elements or parameters is selected from a group consisting of dynamic viscosity, Temperature, Pressure, Reynold's number, expansion, volume flow rate, cross-sectional area and dynamic friction co-efficient, and wherein the deterministic or directional relationship between the elements is defined by a formula or an equation.

13. The method according to claim 10, wherein the zone of potentiality of nodal architecture comprises data having a surrogate relationship between the plurality of elements, and wherein the plurality of elements does not have a direct equation to relate these elements but have a linear relationship between these elements

14. The method according to claim 10, wherein the zone of probability of nodal architecture comprises of data having a probabilistic cause-effect relationship between the elements.

15. The method according to claim 10, wherein the zone of enquiry of nodal architecture comprises of data having a possible cause-effect relationship between the elements, and wherein the cause-effect relationship is not yet defined by any theorem, and wherein the relationship is unknown and is only manifested when the changes are observed between the elements along with the other categories.

16. The method according to claim 10, wherein the building units for the creation of two-dimensional (2D)/three-dimensional (3D) morphology architecture and/or synthetic cognition datascapes generated via the grid generation module includes D-AXONS, D-CELLS and D-TISSUES, and wherein the D-AXONS includes data from individual elements converted into a line, and wherein the line width and colour of the data are mapped to disparate attributes of the data, and wherein the D-CELL is formed when the D-AXONS are arranged in a grid-like pattern in a nodal architecture, and wherein the D-CELL is a holistic data matrix of the system at a single timestamp, and wherein the D-TISSUE is formed as D-CELL timestamps are stacked together to form a large data matrix that captures the whole dynamic or a chronological event of a complex adaptive system, and wherein a plurality of complex adaptive systems stack to form even a larger data matrix of D-ORGAN and D-ORGANISM respectively, and wherein the dynamic relationship between the cells arranged in a 2D or 3D morphology enables to delineate the changes occurring together in which the data is related structurally or functionally.

17. The method according to claim 10, wherein the signature patterns detected by the data mining module include machine learning algorithms and artificial intelligence to identify a result of the analysis of the complex adaptive systems into identifiable categories comprising wellness and illness, harmony and disharmony, best, better, good, bad, worse, and worst, and wherein the datascapes are visualized via data mining module and the changes due to any structural modifications are manifested first and the functional changes follow the structural manifestations, thereby eliminating a need for an understanding through a reductionist approach of studying small functional units individually in order to understand the whole system.

18. The method according to claim 10, wherein the mining and iteration of the datascapes by artificial intelligence and machine learning algorithms includes pattern recognition, image classification, Generative Adversarial Network, Convolutional Neural Network.
